(19) United States
(12) Patent Application Publication
(10) Pub. No.: US 2018/0371008 A1
(43) Pub. Date: Dec. 27, 2018

(54) METHODS OF SYNTHESIZING AND LABELING NUCLEIC ACID MOLECULES

(71) Applicant: LIFE TECHNOLOGIES CORPORATION, Carlsbad, CA (US)

(72) Inventors: Jun LEE, San Diego, CA (US); Weidong ZHENG, Carlsbad, CA (US)

(21) Appl. No.: 16/023,051

(22) Filed: Jun. 29, 2018

Related U.S. Application Data
(63) Continuation of application No. 14/533,029, filed on Nov. 4, 2014, now abandoned.
Application No. 14/533,029 is a continuation of application No. 13/366,769, filed on Feb. 6, 2012, now abandoned.
Application No. 13/366,769 is a continuation of application No. 10/821,128, filed on Apr. 9, 2004, now abandoned.
(60) Provisional application No. 60/461,189, filed on Apr. 9, 2003.

(51) Int. Cl. C07H 21/04; C07H 21/02; C12Q 1/6813; C07H 1/00

(52) U.S. Cl. CPC C07H 21/04; C07H 1/00; C12Q 1/6813; C07H 21/02

(57) ABSTRACT

The present invention is generally related to composition, kits and methods for synthesizing nucleic acid molecules and particularly for synthesizing labeled nucleic acid molecules. Specifically, the invention relates to methods, kits and compositions for synthesizing indirectly labeled nucleic acid molecules. The labeled nucleic acid molecules produced in accordance with the invention are particularly suited as labeled probes for nucleic acid detection, diagnostics, and array analysis.

### BACKGROUND OF THE INVENTION

### Field of the Invention

The present invention relates to methods and materials used to synthesize nucleic acid molecules and particularly to synthesize labeled nucleic acid molecules. The invention also relates to nucleic acid molecules produced by these methods and the use of such nucleic acid molecules in the fields of molecular and cellular biology. The invention also relates to kits and compositions for making and labeling such nucleic acid molecules.

### Background of the Invention

In examining the structure and physiology of an organism, tissue or cell, it is often desirable to determine its genetic content. The genetic framework of an organism is included in the double-stranded sequence of nucleotide bases, the deoxyribonucleic acid (DNA), which is contained in the somatic and germ cells of the organism. The genetic content of a particular segment of DNA, or gene, is only manifested upon production of the protein, which the gene encodes. In order to produce a protein, a complementary copy of one strand of the DNA double helix (“coding strand”) is produced by polymerase enzymes, resulting in a specific sequence of ribonucleic acid (RNA). This particular type of RNA, since it contains the genetic message from the DNA for production of a protein, is called messenger RNA (mRNA).

With any given cell, tissue or organism, there exists myriad mRNA species, each encoding a separate and specific protein. This fact provides a powerful tool to investigators interested in studying genetic expression in a tissue or cell—mRNA molecules may be isolated and further manipulated by various molecular biological techniques, thereby allowing the elucidation of the full functional genetic content of a cell, tissue or organism.

One common approach to the study of gene expression is the production of complementary DNA (cDNA) clones. In this technique, the mRNA molecules from an organism are isolated from an extract of the cells or tissues of the organism. This isolation often employs solid chromatography, such as cellulose or agarose, to which oligomers of Thymine (T) have been complexed. Since the 3′-termini of most eukaryotic RNA molecules contain a string of Adenine (A) bases, and since A binds to T, the mRNA molecules can be rapidly purified from other molecules and substances in the tissue or cell extract. From these purified mRNA molecules, cDNA copies may be made using the enzyme reverse transcriptase (RT), which results in the production of single-stranded cDNA molecules. The single-stranded cDNAs may then be converted into a complete double-stranded DNA copy (i.e., a double-stranded cDNA) of the original mRNA and thus of the original double-stranded DNA sequence encoding this mRNA, contained in the genome of the organism (by the action of a DNA polymerase). The protein-specific double-stranded cDNAs can then be inserted into a plasmid or viral vector, which is then introduced into a host bacterial, yeast, animal or plant cell. The host cells are then grown in culture media, resulting in a population of host cells containing (or in many cases, expressing) the gene of interest.

This entire process, from isolation of mRNA to insertion of the cDNA into a plasmid or vector to growth of host cell populations containing the isolated gene, is termed “cDNA cloning”. If cDNAs are prepared from a number of different mRNAs, the resulting set of cDNAs is called a “cDNA library”. Genotypic analysis of these cDNA libraries can yield much information on the structure and function of the organisms from which they were derived. Moreover, recent breakthroughs in nucleic acid sequencing technology have made possible the sequencing of entire genomes from a variety of organisms, including humans. The potential benefits of a complete genome sequence are many, ranging from applications in medicine to a greater understanding of evolutionary processes. These benefits cannot be fully realized, however, without knowing the types of genes which are expressed with cell function.

Traditionally, functional understanding started with recognizing a functional activity in a cell or tissue, attempting to isolate a protein or proteins associated with that activity, then isolating the gene, or genes, encoding that protein. The isolated protein may also be used to generate antibody reagents. Specific antibodies and fragments of the isolated gene were both employed to study tissue expression and function. Several methods have been used to study protein expression patterns including in situ hybridization studies of tissue sections and northern blots. These methods are both time consuming, labor intensive and expensive.

Recently, new technologies have arisen that allow high throughput expression analysis studies. Through the use of arrays of nucleic acid molecules and labeling technologies, it is possible to determine the expression profile for a number of genes of interest for any cell or tissue, without the need to express and analyze the proteins associated with those genes. Arrays for use in expression analysis are typically made by binding or immobilizing a number of individual genes (or options thereof) to a defined area on a solid support with each different location of the support being associated with a different gene. mRNA molecules or other nucleic acid molecules related to gene expression within a cell or tissue may then be bound or hybridize under appropriate conditions to homologous nucleic acid molecules on the array. In this way, by detecting the association (or hybridization) of nucleic acid molecules related to gene expression of a given cell or tissue with various know genes present in an array, it is possible to determine the expression profile for particular genes for any given cell or tissue type, depending on the content of the array itself.

Arrays or microarrays, depending on the need, may contain hundreds, thousands or even millions of different genetic elements or genes (or portions thereof). Because of the large number of difficult genetic elements that can be included in an array format, array analysis has emerged in the last few years as a flexible method for simultaneously analyzing large numbers of nucleic acid molecules. As noted, arrays consist of a collection of nucleic acid sequences immobilized or bound onto a solid support so that each unique sequence is associated with a defined location or spot or “target”. Numerous types of materials can be used as the solid support in any array and various formats exist. Moreover, various methods are available for depositing nucleic acids onto the array support, depending on the support material. A glass slide is an example of a solid support typically used to construct arrays.

Detection of the interaction between the target nucleic acid on the array and the nucleic acid of the test sample is facilitated by using detectable labels. Typically, the sample that is being analyzed, whether mRNA or DNA or other nucleic acid molecules (or populations thereof), is labeled. Such labeled molecules are commonly called probes. Although various detectable labels are known and can be used in array analysis, fluorescent dyes are the most commonly used label. Particular dyes of interest include the cyanine dyes, such as Cy3 and Cy5.

Since high quality probes are important for successful array analysis, several strategies have been developed for labeling samples used in array analysis. The diversity of labeling methods available can be attributed in part to the availability of different types of labels and the way in which they are used.

To date, one of the most widely used labeling strategies is to convert mRNA populations into a labeled first-strand cDNA population. This is commonly known as “first strand labeling” or “labeling in first-strand synthesis”. In this method, the mRNA transcripts are copied into cDNA molecules with a reverse transcriptase while incorporating a nucleotide modified with a cyanine dye, such as a CyDye nucleotide (Amersham Biosciences). The cDNA synthesis can be primed with a variety of primers including random primers, anchored oligo dT, as well as gene specific primers. In this first-strand method, the incorporation of fluorescently labeled nucleotides is limited by the ability of reverse transcription to efficiently incorporate the labeled nucleotides during nucleic acid synthesis. Because the reverse transcription typically incorporates fluorescently labeled nucleotides less efficiently than unlabeled nucleotides during nucleic acid synthesis, one of the drawbacks of first-strand labeling is that this process tends to reduce yields and produce shorter cDNA molecules, and the ratio of labeled nucleotides incorporated into the synthesized product is lowered. Due to these shortcomings, many practitioners have turned to other methods of labeling cDNA. One popular method relies on labeling the cDNA after nucleic acid synthesis has occurred. In this cDNA post-labeling method, also known as “indirect labeling”, a chemically reactive nucleotide analog, specifically aminoallyl-dUTP, is incorporated into the cDNA during reverse transcription by a reverse transcriptase. This chemically reactive nucleotide analog is then subsequently labeled with, for example, a fluorescent cyanine dye. Because the nucleotide analog is more efficiently incorporated during nucleic acid synthesis (perhaps related to reduced steric hindrance) the amount and length of cDNA product is increased, and the nucleotide analog tends to be more uniformly incorporated at a higher frequency. Accordingly, the post labeling method allows more efficient labeling of probes to be used in various detection methods. However, there is a continuing need for nucleic acid labeling methods and particularly a need for nucleic acid labeling methods to prepare probes used in array analysis. The present invention satisfies this and other needs.

### SUMMARY OF THE INVENTION

The invention provides novel methods for preparing and/or labeling nucleic acid molecules through the use of one or a number of the same or different modified nucleotides that may be labeled with one or a number of the same or different detectable labels. Such labeling can be accomplished in accordance with the invention before or after synthesis of the nucleic acid molecules to be labeled. Through the use of different modified nucleotides and/or different labels, the invention also allows differential labeling of one or a number of the same or different nucleic acid molecules.

Thus, by incorporation of different labels (two or more) into a nucleic acid molecule, the invention may provide more sensitive probes since the different labels have different attributes and characteristics and those different characteristics and attributes can be used to facilitate detection of the probe. In another aspect, different nucleic acid molecules or different populations of nucleic acid molecules may be differentially labeled in accordance with the invention. Thus, by incorporating different labels into different nucleic acid molecules (or populations thereof), the invention provides different probes which can be differentially detected based on the characteristics and features of the labels used. In one example, a population of nucleic acid molecules from one tissue or cell (e.g. mRNA molecules) may be labeled with one detectable label while a second population of nucleic acid molecules from a different cell or tissue may be labeled with a second detectable label. Such differential labeling should allow for simultaneous detection and analysis of multiple nucleic acid samples, thus reducing costs and increasing throughput. For example, a combination of different probes having different labels can be reacted on an array and the gene expression profile can be determined for each different sample based on the label detected.

In yet another aspect, a number (two or more) of different nucleotides may be incorporated into one or a number of different nucleic acid molecules to facilitate labeling of those nucleic acid molecules. In accordance with the invention, the use of multiple (two or more) modified nucleotides during synthesis of a nucleic acid molecule may increase the number of a ratio of incorporated modified nucleotides, labeling such modified nucleotides should therefore provide a probe having a higher amount of ratio of labels, thus producing a more sensitive probe for detection. As noted, such labeling may be accomplished with two or more different detectable labels depending on the need.

The invention also relates to compositions for use in synthesizing one or more nucleic acid molecules and/or labeling one or more nucleic acid molecules. Such compositions may comprise one or a number of the same or different, preferably two or more, modified nucleotides. Such compositions may further comprise one or more single stranded, double stranded or single stranded/double stranded nucleic acid templates (e.g., RNA, DNA, RNA/DNA hybrids etc.), a suitable buffer, one or more nucleotides and/or one or more DNA polymerases or reverse transcriptases. Such compositions may further comprise one or more detectable labels capable of binding to or being coupled to one or more of the modified nucleotides. The compositions of the invention may also comprise one or more solid supports. Reverse transcriptases (RT) in these compositions preferably have RNaseH activity or are reduced or substantially reduced in RNaseH activity, and most preferably are enzymes selected from the group consisting of single sub-unit and multi sub-unit reverse transcriptases and preferably bacterial or viral (preferably retroviral) reverse transcriptases including Molony Murine leukemia virus (MMLV) reverse transcriptase, Rous Sarcoma Virus (RSV) reverse transcriptase, Avian Myeloblastosis Virus (AMV) reverse transcriptase, Rous Associated Virus (RAV) reverse transcriptase, Myeloblastosis Associated Virus (MAV) reverse transcriptase and human immunodeficiency virus (HIV) reverse transcriptase or other Sarcoma-Leukosis Virus (ASLV) reverse transcriptases.

The invention is also directed to methods for making one or more nucleic acid molecules (which are preferably labeled), comprising mixing one or more nucleic acid templates (preferably one or more RNA templates and most preferably one or more messenger RNA templates) and/or with one or more polypeptides or enzymes having DNA dependent polymerase or RNA dependent polymerase activity (including reverse transcriptases and DNA polymerases) and one or more (preferably two or more) modified nucleotides and incubating the mixture under the conditions sufficient to synthesize one or more first nucleic acid molecules complementary to all or a portion of one or more nucleic acid templates. In a preferred aspect, the synthesized nucleic acid molecules contain a number of different (e.g., two or more, three or more, four or more, five or more, six or more, etc.) modified nucleotides. Once a number of the modified nucleotides are incorporated into the synthesized nucleic acid molecules, molecules may be labeled with one or more different detectable labels. In one preferred aspect, the modified nucleotides are labeled by coupling one or more detectable labels to some or all of the modified nucleotides. Preferably, the detectable label is a mono reactive ester form of a free cyanine dye. More preferably, the label is Cy3 or Cy5. In another preferred embodiment, said nucleic acid molecules are labeled by coupling a detectable label to the reactive primary amine of the modified nucleotides incorporated therein. In a preferred embodiment, the one or more first nucleic acid molecules are single-stranded cDNA molecules. The nucleic acid templates suitable for reverse transcription according to this aspect of the invention include any nucleic acid molecule or population of nucleic acid molecules (preferably RNA and most preferably mRNA), particularly those derived from a cell or tissue. In a preferred aspect, a population of mRNA molecules (a number of different mRNA molecules, typically obtained from cells or tissues) are used to make a labeled cDNA library, in accordance with the invention. Preferred cellular sources of nucleic acid templates include bacteria cells, fungal cells, plant cells and animal cells.

The invention is also directed to nucleic acid molecules or labeled nucleic acid molecules produced by methods described herein and to kits comprising these nucleic acid molecules. Such molecules or kits may be used to detect nucleic acid molecules (for example by hybridization), for various diagnostic purposes or microarray analysis. Such molecules may be bound directly or indirectly (for example by hybridization) to one or more solid supports or one or more arrays. The kits containing these nucleic acid molecules also may comprise one or more solid supports or one or more arrays.

The invention is also directed to kits for use in the methods of the invention. Such kits can be used for making labeled nucleic acid molecules. Such kits may comprise one or a number of different (preferably two or more) modified nucleotides (in one or more separate containers). The kits of the invention may also comprise, in the same or different containers, at least one component selected from one or more DNA polymerases (preferably thermostable DNA polymerases), one or more primers, one or more templates, a suitable buffer for nucleic acid synthesis, and one or more nucleotides. The components of the kit may be combined into one or more containers or, alternatively, divided into separate containers. The kits of the invention may also comprise one or more reverse transcriptases which have RNaseH activity or are reduced or substantially reduced in RNaseH activity. Such RTs preferably are selected from the group consisting of the MMLV reverse transcriptase, RSV reverse transcriptase, AMV reverse transcriptase, RAV reverse transcriptase, MAV reverse transcriptase and HIV reverse transcriptase. In additional preferred kits of the invention, the enzymes (reverse transcriptase and/or DNA polymerases) in the containers are present at working conditions. In another aspect, the kits of the invention contain one or more of the same or different labels and preferably the labels are designed to bind or interact with the modified nucleotides or the modified nucleotides incorporated in nucleic acid molecules. In a preferred embodiment, the label is a fluorescent dye. Other preferred embodiments of the present invention will be apparent to one of ordinary skill in light of the following description of the invention, and of the claims.

### BRIEF DESCRIPTION OF THE FIGURES

FIG. 1 shows a general method of synthesizing and indirectly labeling nucleic acid molecules.

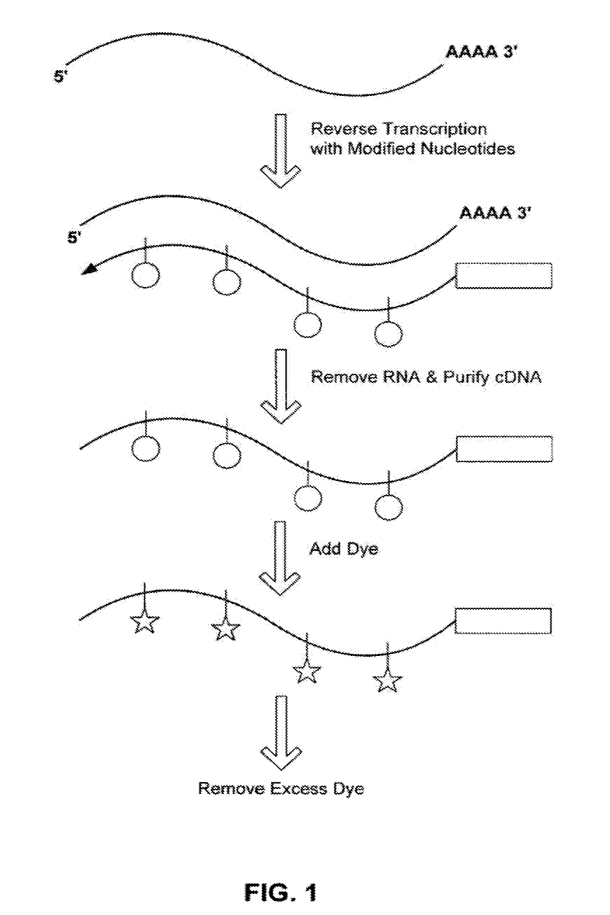

FIG. 2 shows a flow chart showing a preferred method of synthesizing and indirectly labeling nucleic acid molecules.

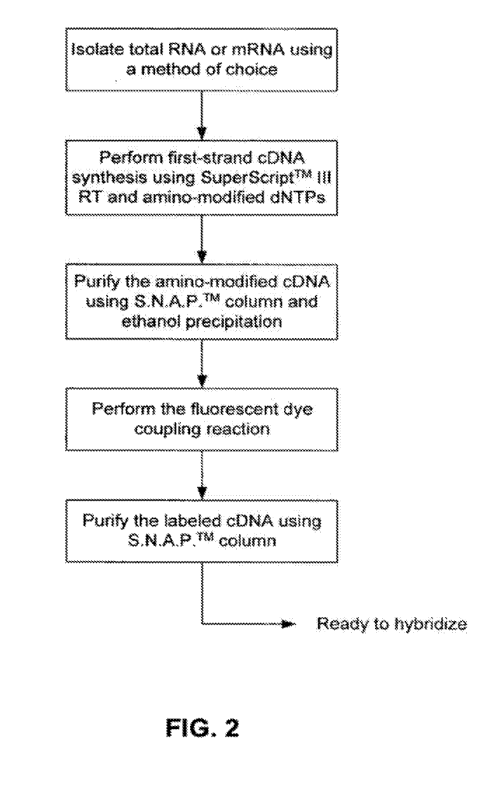

### DETAILED DESCRIPTION OF THE INVENTION

### Definitions

In the descriptions that follow, a number of terms used in recombinant DNA technology is utilized extensively. In order to provide a clear and more consistent understanding of the specification and claims, including the scope to be given such terms, the following definitions are provided:

Primer. As used herein “primer” refers to a single-stranded oligonucleotide that is extended by covalent bonding of nucleotide monomers during amplification or polymerization of a nucleic acid molecule.

Template. The term “template” as used herein refers to double-stranded or single-stranded nucleic acid molecules which are to be amplified, synthesized or sequenced. In the case of a double-stranded molecule, the denaturation of the strands to form a first and second strand is preferably performed before these molecules may be amplified, synthesized or sequenced, or the double-stranded molecule may be used directly as a template. For single-stranded templates, at least one primer, complementary to a portion of the template is hybridized under appropriate conditions and one or more polymerases or reverse transcriptases may synthesize a nucleic acid molecule complementary to all or a portion of said template. The newly synthesized molecules, according to the invention, may be equal or shorter in length than the original template.

Incorporating. The term “incorporating” as used herein means become a part of a DNA and/or RNA molecule or primer.

Nucleotide. As used herein “nucleotide” refers to a base-sugar-phosphate combination. Nucleotides may include monomeric units of a nucleic acid sequence (DNA and RNA). The term nucleotide includes ribo- and deoxy-nucleoside monophosphates, diphosphates, and/or triphosphates and derivatives thereof. The term includes ribonucleoside triphosphates such as ATP, UTP, CTP, GTP and deoxyribonucleoside triphosphates such as dATP, dCTP, dITP, dUTP, dGTP, dTTP, or derivatives thereof. Such derivatives include, for example, [αS]dATP, 7-deaza-dGTP and 7-deaza-dATP, and nucleotide derivatives that confer nuclease resistance on the nucleic acid molecule containing them. The term nucleotide as used herein also refers to dideoxyribonculeoside triphosphates (ddNTPs) and their derivatives. Illustrated examples of dideoxyribonucleoside triphosphates include, but are not limited to, ddATP, ddCTP, ddGTP, ddITP, and ddTTP. The term nucleotide as used herein also refers to deoxyribonucleoside monophosphates and ribonucleoside monophosphates such as dAMP, dGMP, dCMP, dTMP, dUMP, AMP, GMP, CMP, UMP, and their derivatives. Nucleotide also includes ribonucleoside diphosphates and deoxyribonucleoside diphosphates such as dADP, dGDP, dCDP, dTDP, dUDP, ADP, GDP, CDP, UDP and their derivatives. The term nucleotide also refers to nucleotides with oxidized, alkylated or methylated bases, for example thymine glycol, 8-oxoguanine, 4,6-diamino-5-formamidopyrimidine, urea, 3-methyladenine, 7-methyl-guanine, or 6-methylguanine. According to the present invention, a nucleotide may be unlabeled or detectably labeled by well-known techniques. Detectable labels include, for example, radioactive isotopes, fluorescent labels, chemiluminescent labels, bioluminescent labels and enzyme labels.

Modified Nucleotide. As used herein “modified nucleotide” refers to any molecule (preferably a chemical compound) that can be incorporated in a nucleic acid molecule during nucleic acid synthesis or any (i) molecule that can otherwise function as a nucleotide in a nucleic acid molecule or sequence and (ii) has the ability to bind (covalently, non-covalently, directly or indirectly) with or to one or more labels, preferably through one or more reactive groups located on the modified nucleotides. A modified nucleotide can be any nucleotide having one or more modifications, including any type of modification in any location or number of locations within the nucleotide. Such modification or modifications may be included in the base, sugar or phosphate structures (or combinations thereof) and a modification can change the characteristics or structure or function of one or a number of elements of the nucleotide. Modifications can include addition of one or more molecules or chemical groups, substitution of one or more molecules or chemical groups with other molecules or chemical group, and/or deletion of one or more molecules or chemical groups or conjugate. Preferably, a modified nucleotide does not contain a detectable label or are unlabeled, and are preferably not bound or conjugated or complexed to a detectable label prior to incorporation of the modified nucleotide in a nucleic acid molecule. In any event, the modified nucleotide can be labeled (or bound or complexed with or to one or more labels) prior to or after such modified nucleotide are incorporated into a nucleic acid molecule. In one aspect, a modified nucleotide does not contain or lacks (or is not interacted with or not bound to) a fluorophore and/or a fluorescent dye and/or a fluorescent moiety, although such molecules may be added once the modified nucleotide is incorporated in a nucleic acid molecule. In another aspect, a modified nucleotide has not been interacted with a fluorophore and/or a fluorescent dye and/or a fluorescent moiety prior to incorporation of the modified nucleotide into a nucleic acid molecule. In other aspects, a modified nucleotide is not complexed or attached to other specified labels, bioluminescent labels and enzyme labels or combinations thereof. The modified nucleotides include, but are not limited to, nucleotides containing one or more reactive groups such as primary amine, hydroxyl, sulfhydryl, aldehyde, or carboxylate Group. Examples of modified nucleotides of the invention include for example, aminoallyl-dUTP (AA-dUTP), aminohexyl-dATP (AH-dATP), aminoallyl-dCTP (AA-dCTP), as well as those disclosed in Folsom et al., Anal. Biochem. 82(2):309-314 (Nov. 1, 1989); GebeyeHu et Nuc. Acid Res. 15(11):4513-4534 (1987); ZoFall et al., Nucl. Acid Res. 28(21):4382-4390 (2000).

Oligonucleotide. “Oligonucleotide” refers to a synthetic or natural molecule such as a nucleic acid molecule comprising a covalently linked sequence of a number of nucleotides and/or modified nucleotides which are joined by a phosphodiester bond between the 3′ position of the deoxyribose or ribose of one nucleotide and the 5′ position of the deoxyribose or ribose of the adjacent nucleotide.

Amplification. As used herein “amplification” refers to any in vitro method for increasing the number of copies of a nucleotide sequence with use of a polymerase. Nucleic acid amplification results in the incorporation of nucleotides into a DNA and/or RNA molecule or primer thereby forming a new molecule complementary to all or a portion of the template. The formed nucleic acid molecule and its template can be used as templates to synthesize additional nucleic acid molecules. As used herein, one amplification reaction may consist of many rounds of replication. DNA amplification reactions include, for example, polymerase chain reactions (PCR). One PCR reaction may consist of 5 to 100 “cycles” of denaturation and synthesis of a DNA molecule.

Hybridization. The terms “hybridization” and “hybridizing” refers to base paring of two complementary single-stranded nucleic acid molecules (RNA and/or DNA) to give a double-stranded molecule. As used herein, two nucleic acid molecules may be hybridized although the base paring is not completely complementary. Accordingly, mismatched bases do not prevent hybridization of two nucleic acid molecules provided that appropriate conditions, well known in the art, are used.

Probe. The term “probe” refers to one or more nucleic acid molecules (single or double-stranded) nucleotides that are detectably labeled with one or more detectable markers or labels. Such labels or markers may be the same or different and may include radioactive labels, fluorescent labels, chemiluminescent labels, bioluminescent labels and enzyme labels, although one or more fluorescent labels (which are the same or different) are preferred in accordance with the invention. Probes in accordance with the invention may be used in the detection of nucleic acid molecules by hybridization and thus may be used in diagnostic assays or for microarray analysis.

Detectable Labels. A “detectable label” or “detectably labeled” or “label” or “labeled” as used herein refers to any molecule or moiety or composition or complex which can be determined to be present in a sample of interest or otherwise detected by one or a number of means of detection well known in the art. Such detection may be accomplished by visualization, fluorescence spectrometers, absorption spectrometers, fluorescence microscopes, transmission light microscopes, flow cytometers. Fiber optic sensors, and immunoassay instruments. Chemical analysis methods can include infrared spectrometry, NMR spectrometry, absorption spectrometry, fluorescence spectrometry, mass spectrometry and chromatographic methods. Such labels may be complexed with or linked or bound to any compound or element to allow detection of the labeled compound or element. Detectable labels include, but are not limited to, fluorescent labels (including fluorophores), radioactive isotopes, chemiluminescent labels, bioluminescent labels, and enzyme labels.

For the purpose of the present invention, a fluorophore can be a substance which itself fluoresces, or a substance that fluoresces in particular situations (e.g., when in proximity to another fluorophore, as occurs in FRET). The term “fluorophore” or “fluor” is meant to encompass fluorescent moieties that may be linked (covalently or non-covalently or indirectly or directly) to another molecule, as well as free fluorescent molecules. Molecules that become: fluorescent only after attachment to another molecule, such as a peptide or nucleic acid, are also within the scope of the invention.

In principle, any fluorophore now known, or later discovered, can be used in accordance with the methods, compositions and kits of the present invention. In certain embodiments, fluorophores suitable for use in the present invention include those that are excitable at, and/or emit fluorescence at, a wavelength falling within the range of wavelengths from about 200 nm to about 800 nm; from about 250 nm to about 800 nm; from about 250 nm to about 750 nm; from about 300 nm to about 700 nm; from about 350 nm to about 650 nm; from about 400 nm to about 600 nm; from about 450 nm to about 600 nm; from about 450 nm to about 580 nm; from about 450 nm to about 575 nm; from about 450 nm to about 570 nm; from about 500 nm to about 600 nm; from about 500 nm to about 590 nm; from about 500 nm to about 580 nm; from about 500 nm to about 575 nm; from about 500 nm to about 570 nm; and the like. As one of ordinary skill will readily appreciate, any fluorophore with an excitation maximum and an emission maximum within the recited ranges is suitable for use in accordance with the present invention, whether or not the actual, specific excitation and emission maxima for that given fluorophore are specifically set forth above.

In view of the availability of an array of appropriate compounds, it is well within the capabilities of one skilled in the art to choose a reactive fluorescent molecule or set of molecules that is appropriate to the practice of the present invention, given the above-noted guidelines for excitation and emission maxima. Many appropriate fluorophores are commercially available from sources such as Molecular Probes Inc. (Eugene, Oreg.).

Many of these methods are quite appropriate for use in preparing the various compounds required to practice the present invention. One skilled in the art will be able, without undue experimentation, to choose a suitable method for preparing a desired fluorescently labeled nucleic acid, oligonucleotide or the like. See, for example, Protocols for Oligonucleotide Conjugates, Vol. 26 of Methods in Molecular Biology, Agrawal, ed., Humana Press, Totowa, N.J. (1994). Additionally, as the art of organic synthesis, particularly in the area of nucleic acid chemistry, continues to expand in scope new methods will be developed which are equally as suitable as those now known. The following discussion is offered as representative of the array of compounds and techniques that can be used to modify nucleic acids. Methods useful in conjunction with the present invention are not to be construed as limited by this discussion.

Fluorescent moieties and molecules useful in practicing the present invention include but are not limited to fluorescein, rhodamine, coumarin, dimethylaminonaph-thalene sulfonic acid (dansyl), pyrene, anthracene, nitrobenz-oxadiazole (NBD), acridine and dipyrrometheneboron difluoride and derivatives thereof. More specifically, non-limiting examples of fluorescent moieties and molecules useful in practicing the present invention include, but are not limited to:

carbocyanine, dicarbocyanine, merocyanine and other cyanine dyes (e.g., CyDye™ fluorophores, such as Cy3, Cy3.5, Cy5, Cy5.5 and Cy7 from Pharmacia). These dyes have a maximum fluorescence at a variety of wavelengths: green (506 nm and 520 nm), green-yellow (540 nm), orange (570 nm), scarlet (596 nm), far-red (670 nm), and near infrared (694 nm and 767 nm);

coumarin and its derivatives (e.g., 7-amino-4-methylcoumarin, aminocoumarin and hydroxycoumarin);

BODIPY dyes (e.g., BODIPY FL, BODIPY 630/650, BODIPY 650/665, BODIPY TMR);

fluorescein and its derivatives (e.g., fluorescein isothiocyanate);

rhodamine dyes (e.g. rhodamine green, rhodamine red, tetramethylrhodamine, rhodamine 6G and lissamine rhodamine B);

Alexa dyes (e.g., Alexa Fluor-350, -430, -488, -532, -546, -568, -594, -663 and -660, from Molecular Probes);

fluorescent energy transfer dyes (e.g., thiazole orange-ethidium heterodimer, TOTAB, etc.);

proteins with luminescent properties, e.g.: green fluorescent protein (GFP) and mutants and variants thereof, including by way of non-limiting example fluorescent proteins having altered wavelengths (e.g., YFP, RFP, etc.). See Chiesa et al., Biochem. J. 355:1-12 (2001). Recombinant aequorin and green fluorescent protein as valuable tools in the study of cell signaling. Sacchetti et al., Biochem. J. 355:1-12 (2000). The molecular determinants of the efficiency of green fluorescent protein mutants. Larrick, J. W. et al., Histol Histopathol. 15:101-107 (1995). Green fluorescent protein: untapped potential in immunotechnology. Larrick, J. W. et al., Immunotechnology 1:83-86 (1995).

aequorin and mutants and variants thereof;

DsRed protein (Baird et al., Biochemistry, mutagenesis, and oligomerization of DsRed, a red fluorescent protein from coral. Proc Natl. Acad. Sci. USA 97:11984-11989 (2000)), and mutants and variants thereof (see Verkhusha et al., 2001. An enhanced mutant of red fluorescent protein DsRed for double labeling and developmental timer of neural fiber bundle formation. J. Biol. Chem. 276:29621-29624 (2001); Bevis, B. J. and Glick, B. S., Rapidly maturing variants of the Discosoma red fluorescent protein (DsRed). Nat. Biotechnol. 20:83-87 (2002); Terskikh et al., Analysis of DsRed Mutants. Space around the fluorophore accelerates fluorescence development. J. Biol. Chem. 277:7633-7636 (2002); Campbell et al., A monomeric red fluorescent protein. Proc Natl Acad. Sci. USA 99:7877-7882 (2002); and Knop et al., Improved version of the red fluorescent protein (drFP583/DsRed/RFP). Biotechniques 33:592, 594, 596-598 (2002)); and

other fluors, e.g., 6-FAM, HEX, TET, F12-dUTP, L5-dCTP, 8-anilino-1-napthalene sulfonate, pyrene, ethenoadenosine, ethidium bromide prollavine monosemicarbazide, p-terphenyl, 2,5-diphenyl-1,3,4-oxadiazole, 2,5-diphenyloxazole, p-bis[2-(5-phenyloxazolyl)]benzene, 1,4-bis-2-(4-methyl-5-phenyloxazolyl)-benzene, lanthanide chelates, Pacific blue, Cascade blue, Cascade Yellow, Oregon Green, Marina Blue, Texas Red, phycoerythrin, eosins and erythrosins;

as well as derivatives of any of the preceding molecules and moieties. Fluorophores, and kits for attaching fluorophores to nucleic acids and peptides, are commercially available from, e.g., Molecular Probes (Eugene, Oreg.) and Sigma/Aldrich (St. Louis, Mo.).

Fluorescent moieties useful in practicing the present invention can be attached to any location on a nucleic acid, including sites on the base segment and sites on the sugar segment. Thus, the fluorophore may be covalently attached to a nucleic acid at a position selected from the group consisting of the 3′-terminus, the 5′-terminus, an internal position and combinations thereof. See, generally, Goodchild, Bioconjug. Chem. 1:165-187 (1990). Although any suitable fluorophore can be associated with an oligonucleotide, some of the more commonly used examples are fluorescein, tetramethylrhodamine, Texas Red and Lissamine rhodamine B.

A number of techniques have been developed for converting specific constituents of DNA and RNA strands into fluorescent adducts. For a review, see, Leonard and Tolman, in “Chemistry, Biology and Clinical Uses of Nucleoside Analogs,” A. Bloch, ed., Ann. N.Y. Acad. Sci. 255:43-58 (1975).

Chemical methods are available to introduce fluorescence into specific nucleic acid bases. For example, reaction of chloracetaldehyde with adenosine and cytidine yields fluorescent products. The reaction can be controlled with respect to which of the two bases is derivatized by manipulating the pH of the reaction mixture; the reaction at 37° C. proceeds rapidly at the optimum pH of 4.5 for adenosine and 3.5 for cytidine (Barrio et al., Biochem. Biophys. Res. Commun. 46:597-604 (1972)). This reaction is also useful for rendering fluorescent the deoxyribosyl derivatives of these bases (Kochetkov et al., Dokl. Akad. Nauk. SSSR C 213:1327-1330 (1973)).

DNA and RNA can be modified by reacting their cytidine residues with sodium bisulfite to form sulfonate intermediates that are then coupled to reactive nitrogen compounds such as hydrazides or amines (Viscidi et al., J. Clin. Microbiol. 23:311 (1986); and Draper and Gold, Biochemistry 19:1774 (1980)). RNA can also be labeled at the 3′ terminus by selective oxidation. The selective oxidation of the 3′ ribose of RNA by periodate yields a dialdehyde which can then be coupled with an amine or hydrazide reagent (Churchich, Biochim. Biophys. Acta 75:274-276 (1963); Hileman et al., Bioconjug. Chem. 5:436-444 (1994)).

Individual nucleotides can be derivatized with fluorescent moieties on the base or sugar components. Modification to the base can occur at exocyclic amines or at the carbons of the ring. See, for example, Levina et al., Bioconjug Chem. 4:319-325 (1993). Modification of the sugar moiety can take place at the oxygens of the hydroxyl groups or the carbon atoms of the ribose ring. See, for example, Augustyns et al., Nucleic Acids Symp. Ser. 24:224 (1991); Yamana et al., Bioconjug Chem. 7:715-720 (1996); Guzaev et al., Bioconjug. Chem. 5:501-503 (1994); and Ono et al., Bioconjug. Chem. 4:499-508 (1993), and references contained within, the disclosure of each of which is incorporated herein by reference.

The modified labeled nucleic acids can also be 2′-deoxyribonucleic acids which are labeled at the 3′-hydroxyl via, for example, alkylation or acylation. These labeled nucleic acids will function like dideoxynucleic acids, terminating synthesis, when used in the Sanger method.

Fluorescent G derivatives have also been prepared from the natural base upon its reaction with variously substituted malondialdehydes. See, Leonard and Tolman, in “Chemistry, Biology and Clinical Uses of Nucleoside Analogs,” A. Bloch, ed., Ann. N.Y. Acad. Sci. 255:43-58 (1975).

In addition to the various methods for converting the bases of an intact oligonucleotide into their fluorescent analogs, there are a number of methods for introducing fluorescence into an oligonucleotide during its de novo synthesis.

Generally, at least three methods are available for fluorescent tagging a synthetic oligonucleotide. These methods utilize fluorescently-tagged supports, fluorescently-tagged 5′ modification reagents and fluorescently-tagged monomers.

The first of these methods utilizes a fluorescently-tagged linker that tethers the oligonucleotide strand to the solid support. When the oligonucleotide strand is cleaved from the solid support, the fluorescent tether remains attached to the oligonucleotide. This method affords an oligonucleotide that is fluorescently labeled at its 3′-end. In a variation on this method, the 3′-end of the nucleic acid is labeled with a linker that bears an amine, or other reactive or masked reactive group, which can be coupled to a reactive fluorophore following cleavage of the oligonucleotide from the solid support. This method is particularly useful when the fluorophore is not stable to the cleavage or deprotection conditions.

Detectable labels include, for example, radioactive isotopes, fluorescent labels, chemiscent labels, bioluminescent labels and enzyme labels. Fluorescent labels of nucleotides may include but are not limited to fluorescene, 5-carboxyfluorescene (FAM), 2′7′-dimethoxi-4′5-dicloro-6-carboxyfluorescene (JOE), rhodamine, 6-carboxyrhodamine (R6G), M, N, N′, N′-tetrametal-6-carboxyrhodamine (TAMRA), 6-carboxy-X-rhodamine (ROX), 4-(4′ dimethylaminophenylazo) benzoic acid (DABCYL) cascade blue, Oregon green, Texas red, Cyane and 5-(2′-aminoethyl) aminonapthalene-1 sulphanic acid (EDANS). Specific examples of the mono reactive ester form of free cyanine dyes which may be coupled to the reactive primary amine of a modified nucleotide, include, but are not limited to, Cy3 and Cy5 dyes (Amersham Bio Sciences Inc., Piscataway, N.J.).

Microarray. “Arrays” and “mircroarrays” are well known in the art. Methods of creating arrays are also well known, including printing on a solid surface using pins (passive pins, quill pins, and the like) or spotting with individual drops of solution. Passive pins draw up enough sample to dispense a single spot. Quill pins draw up enough liquid to dispense multiple spots. Bubble printers use a loop to capture a small volume which is dispensed by pushing a rod through the loop. Microdispensing uses a syringe mechanism to deliver multiple spots of a fixed volume. In addition, solid supports, can be arrayed using piezoelectric (ink jet) technology, which actively transfers samples to a solid support.

One method is described in Shalon and Brown (WO 95/35505, published Dec. 28, 1995) which is incorporated herein by reference in its entirety. The method and apparatus described in Shalon and Brown can create an array of up to six hundred spots per square centimeter on a glass slide using a volume of 0.01 to 100 nl per spot. Suitable concentrations of antibody range from about 1 ng/μl. to about 1 μg/μl. In the present invention, each spot can contain one or more than one distinct antibody.

Other methods of creating arrays are known in the art, including photolithographic printing, (Pease, et al, Proc. Natl. Acad. Sci. USA 91(11):5022-5026 (1994)) and in situ synthesis. While known in situ synthesis methods are less useful for synthesizing, polypeptides long enough to be antibodies, they can be used to make polypetides up to 50 amino acids in length, which can serve as binding proteins as described below.

The microarrays can be created on a variety of solid supports such as plastics (e.g. polycarbonate), complex carbohydrates (e.g. agarose and SEPHAROSE™), acrylic resins (e.g. polyacrylamide and latex beads), and nitrocellulose. Preferred solid support materials include glass slides, silicon wafers, and positively charged nylon.

### Overview

The present invention provides kits, compositions and methods useful in overcoming the labeling limitations often observed during direct labeling of nucleic acid molecules. Thus, the invention facilitates the production of labeled nucleic acid molecules (particularly cDNA molecules) not heretofore possible.

In general, the invention provides compositions for use in the synthesis of one or more nucleic acid molecules which may be subsequently coupled to one or more detectable labels. Such compositions may comprise one or a number (preferably three or more, four or more, five or more, six or more etc.) two or more, modified nucleotides. In a preferred embodiment, the modified nucleotides have reactive primary amines. In a more preferred embodiment, at least two of the modified nucleotides are selected from the group consisting of AA-dUTP and AH-dATP.

Compositions of the present invention may also comprise one or more reverse transcriptases (preferably viral or retroviral reverse transcriptases and preferably one or more single or multi-subunit reverse transcriptases). The enzymes in these compositions are preferably present in working concentrations and have RNase H activity or are reduced or substantially reduced in RNase H activity, although mixtures of enzymes, some having RNase H activity and some reduced or substantially reduced in RNase H activity, may be used in the compositions of the invention. Preferred reverse transcriptase includes MMLV reverse transcriptases, HIV reverse transcriptases or Avain Sarcome-Leukosis Virus (ASLV) reverse transcriptases and mutants, fragments or derivatives thereof. ASLV reverse transcriptases includes but is not limited to Rous Sarcoma Virus (RSV) reverse transcriptase, Avian Myeloblastosis Virus (AMV) reverse transcriptase, Avian Erythroblastosis Virus (AEV) Helper Virus MCAV reverse transcriptase, Avian Myelocytomatosis Virus MC29 Helper Virus MCAV reverse transcriptase, Avian Reticuloendotheliosis Virus (REV-T) Helper Virus REV-A reverse transcriptase, Avian Sarcoma Virus UR2 Helper Virus UR2AV reverse transcriptase.

The invention is also directed to methods for reverse transcription of one or more nucleic acid molecules comprising mixing one or more nucleic acid templates, which is preferably RNA or messenger RNA (mRNA) and more preferably a population of mRNA molecules, with one or more polypeptides having DNA polymerase activity and/or reverse transcriptases activity (preferably single or multi-subunit RTs) and incubating the mixture under conditions sufficient to make one or more nucleic acid molecules complementary to all or a portion of the one or more templates. To make the nucleic acid molecule or molecules complementary to the one or more templates, at least one primer (e.g., an oligo(dT) primer), one or more nucleotides (at least some of which are the same or different modified nucleotides), and one or more suitable nucleic acid synthesis buffers may be preferably used for nucleic acid synthesis. Such synthesis using one or more modified nucleotides provides for one or more nucleic acid molecules having one or a number of the same or different modified nucleotides incorporated in the synthesized nucleic acid sequence. The synthesized molecules containing the modified nucleotides can then be labeled with one or more of the same or different labels, preferably by reacting one or more labels with the same or all of the modified nucleotides incorporated in the synthesized one or more nucleic acid molecules. Thus, the invention allows production of one or more labeled nucleic acid molecules. In accordance with the invention, the amount of labeled product is preferably measured based on percent incorporation of the label of interest into synthesized product as may be determined by one skilled in the art and as discussed in the Examples, although other means of measuring the amount or efficiency of labeling of product will be recognized by one of ordinary skill in the art. The invention provides for enhanced or increased percent binding of label after synthesis of a nucleic acid molecule from a template, preferably after synthesis of one or more cDNA molecules from RNA. According to the invention, such enhancement or increase in percent binding is preferably about equal to or greater than a 2-fold, a 5-fold, a 10-fold, a 15-fold, a 20-fold, a 25-fold, a 30-fold, a 40-fold, a 50-fold increase, or a 100 fold increase or enhancement in percent binding compared to a standard indirect labeling procedure using only one modified nucleotide, i.e., AA-dUTP. In preferred embodiments, the enhancement or increase in percent binding is preferably 2-5 fold, 2-10 fold, 2-15 fold, 2-20 fold, 2-25 fold, 2-30 fold, 2-35 fold, 2-40 fold, 2-50 fold, 2-60 fold, 2-70 fold, 2-80 fold, 2-90 fold, 2-100 fold. In another aspect, the percent binding of labeled nucleotide (preferably a fluorescently label) after synthesis is equal to or greater than about 5%, equal to or greater than about 7.5%, equal to or greater than about 10%, equal to or greater than about 15%, equal to or greater than about 20%, equal to or greater than about 25% equal to or greater than about 30%, equal to or greater than about 40%, equal to or greater than about 50%, equal to or greater than 60%, equal to or greater than 70%, equal to or greater than 80%, equal to or greater than 90%, or equal to or greater than 100% . Nucleic acid templates suitable for reverse transcription according to this aspect of the invention include any nucleic acid molecule, particularly those derived from a prokaryotic or eukaryotic cell. Such cells may include normal cells, diseased cells, transformed cells, established cells, progenitor cells, precursor cells, fetal cells, embryonic cells, bacterial cells, yeast cells, animal cells (including human cells), avian cells, plant cells and the like, or tissue isolated from a plant or an animal (e.g., human, cow, pig, mouse, sheep, horse, monkey, canine, feline, rat, rabbit, bird, fish, insect, etc.). Such nucleic acid molecules may also be isolated from viruses.

The invention also provides labeled nucleic acid molecules and/or nucleic acid molecules comprising one or more modified nucleotides produced according to methods described herein. Such nucleic acid molecules may be single or double stranded and are useful as detection probes and/or for array analysis. Depending on the detectable labels used, the labeled molecules may contain one or a number of labels. Where multiple labels are used, the molecules may comprise a number of the same or different labels. Thus, one type or multiple different detectable labels may be used to provide for the labeled nucleic acid molecules of the invention. Such labeled nucleic acid molecules will thus comprise one or more labeled nucleotides (which may be the same or different). The nucleic acid molecules of the invention may also comprise one or more modified nucleotides (which may be the same or different) and/or one or more labels (which may be the same or different).

The invention also provides kits for use in accordance with the invention. Such kits may comprise a carrier means, such as a box or carton, having in confinement therein one or more container means, such as vials, tubes, bottles and the like, wherein the kit comprises, in the same or different containers, one or more (preferably two or more) of the same or different modified nucleotides. The kits of the invention may also comprise, in the same or different containers, one or more DNA polymerases, one or more primers, one or more suitable buffers and/or one or more nucleotides such as deoxynucleocide triphosphates (dNTPs), one or more reverse transcriptases, one or more detectable labels, one or more solid supports, and/or one or more arrays.

### Sources of Enzyme

Reverse transcriptases for use in the invention include, but are not limited to, retroviral reverse transcriptase, retrotransposon reverse transcriptase, hepatitis B reverse transcriptase, cauliflower mosaic virus reverse transcriptase (CMV-RT), bacterial reverse transcriptase, Rausher Leukemia Virus (RLV-RT), Mouse Mammary Tumor Virus (MMTV-RT), Tobacco Mosaic Virus (TMV-RT), Human Foamy Virus HMV-RT), Tth DNA polymerase, Taq DNA polymerase (Saiki, R X, et al., Science 239:487-491 (1988); U.S. Pat. Nos. 4,889,818 and 4,965,188), Tne DNA polymerase (PCT Publication No. WO 96/10640), Tma DNA polymerase (U.S. Pat. No. 5,374,553) and mutants, fragments, variants or derivatives thereof (see, e.g., commonly owned U.S. Pat. Nos. 5,948,614 and 6,015,668, which are incorporated by reference herein in their entireties). Preferably, reverse transcriptases for use in the invention include retroviral reverse transcriptases such as M-MLV reverse transcriptase, AMV reverse transcriptase, HIV reverse transcriptases, RSV reverse transcriptase, RAV reverse transcriptase, MAV reverse transcriptase, and generally ASLV reverse transcriptases. Additional reverse transcriptases which may be used to prepare compositions of the invention include bacterial reverse transcriptases (e.g., Escherichia coli reverse transcriptase) (see, e.g., Mao et al., Biochem. Biophys. Res. Commun. 227:489-93 (1996)) and reverse transcriptases of Saccharomyces cerevisiae (e.g., reverse transcriptases of the Ty1 or Ty3 retrotransposons) (see, e.g., Cristofari et al., Jour. Biol. Chem. 274.36643-36648 (1999); Mules et al., Jour. Virol. 72:6490-6503 (1998)). As will be understood by one of ordinary skill in the art, modified reverse transcriptases or modified DNA polymerases may be obtained by recombinant or genetic engineering techniques that are routine and well-known in the art. Mutant reverse transcriptases or mutant DNA polymerases can, for example, be obtained by mutating the gene or genes encoding the reverse transcriptase or DNA polymerase of interest by site-directed or random mutagenesis. Such mutations may include point mutations, deletion mutations and insertional mutations. For example, one or more point mutations (e.g., substitution of one or more amino acids with one or more different amino acids) may be used to construct mutant reverse transcriptases or DNA polymerases for use in the invention.

Preferred enzymes for use in the invention include those that are reduced or substantially reduced or lacking in RNase H activity. Such enzymes that are reduced or substantially reduced or lacking in RNase H activity may be obtained by mutating, for example, the RNase H domain within the reverse transcriptase of interest, for example, by introducing one or more (e.g., one, two, three, four, five, ten, twelve, fifteen, twenty, thirty, etc.) point mutations, one or more (e.g., one, two, three, four, five, ten, twelve, fifteen, twenty, thirty, etc.) deletion mutations, and/or one or more (e.g., one, two, three, four, five, ten, twelve, fifteen, twenty, thirty, etc.) insertion mutations as described above.

An enzyme “reduced in RNase H activity” has any detectable reduction (for example, 1% or greater) in RNase H activity compared to the corresponding wild-type or RNase H⁺ enzymes. By an enzyme “substantially reduced in RNase H activity” is meant that the enzyme has less than about 30%, less than about 25%, less than about 20%, more preferably less than about 15%, less than about 10%, less than about 7.5%, or less than about 5%, and most preferably less than about 5% or less than about 2%, of the RNase H activity of the corresponding wild-type or RNase H⁺ enzyme, such as wild-type Moloney Murine Leukemia Virus (M-MLV), Avian Myeloblastosis Virus (AMV) or Rous Sarcoma Virus (RSV) reverse transcriptases.

Reverse transcriptases having reduced or substantially reduced or lacking RNase H activity have been previously described (see U.S. Pat. No. 5,668,005, U.S. Pat. No. 6,063,608, and PCT Publication No. WO 98/47912). The RNase H activity of any enzyme may be determined by a variety of assays, such as those described, for example, in U.S. Pat. No. 5,244,797, in Kotewicz, M. L., et al., Nucl. Acids Res. 16:265 (1988), in Gerard, G. F., et al., FOCUS 14(5):91 (1992), and in U.S. Pat. No. 5,668,005, the disclosures of all of which are fully incorporated herein by reference. Enzymes “lacking” in RNase H activity shall mean the RNase H activity is undetectable by the gel assay and/or the solubilization assay described in U.S. Pat. No. 5,668,005. Preferred enzymes for use the invention include Superscript™, Superscript II™, Thermoscript™, Fluoroscript™, M-MLV Reverse Transcriptase, and AMV Reverse Transcriptase, all available from Invitrogen Corporation.

Particularly preferred enzymes for use in the invention include, but are not limited to, M-MLV RNase H minus reverse transcriptase, RSV RNase H minus reverse transcriptase, AMV RNase H minus reverse transcriptase, RAV RNase H minus reverse transcriptase, MAV RNase H minus reverse transcriptase and HIV RNase H minus reverse transcriptase. It will be understood by one of ordinary skill, however, that any enzyme capable of producing a DNA molecule from a ribonucleic acid molecule (i.e., having reverse transcriptase activity) that is reduced or substantially reduced in RNase H activity may be equivalently used in the compositions, methods and kits of the invention.

In additional aspects, thermostable reverse transcriptases are used in the invention, which retain at least about 50%, at least about 60%, at least about 70%, at least about 85%, at least about 95%, at least about 97%, at least about 98%, at least about 99%, at least about 100% of reverse transcriptase activity after heating to 50° C. for 5 minutes.

Enzymes for use in the invention also include modified or mutated reverse transcriptase (e.g., a modified or mutated retroviral reverse transcriptase) having a reverse transcriptase activity that has a half-life of greater than that of the corresponding unmodified or un-mutated reverse transcriptase at an elevated temperature, i.e., greater than 37° C. In some embodiments, the half-life of a reverse transcriptase of the present invention may be 5 minutes or greater and preferably 10 minutes or greater at 50° C. In some embodiments, the reverse transcriptases of the invention may have a half-life at 50° C. equal to or greater than about 25 minutes, preferably equal to or greater than about 50 minutes, more preferably equal to or greater than about 100 minutes, and most preferably, equal to or greater than about 200 minutes at 50° C. In some embodiments, the reverse transcriptases of the invention may have a half-life at 50° C. that is from about 10 minutes to about 200 minutes, from about 10 minutes to about 150 minutes, from about 10 minutes to about 100 minutes, from about 10 minutes to about 75 minutes, from about 10 minutes to about 50 minutes, from about 10 minutes to about 40 minutes, from about 10 minutes to about 30 minutes, or from about 10 minutes to about 20 minutes. Reverse transcriptases which exhibit increased thermostability are described in U.S. application Ser. No. 09/845,157, filed May 1, 2001 and PCT Publication No. WO 01/92500 (the entire disclosure of which is incorporated herein by reference).

Enzymes for use in the invention also include those in which terminal deoxynucleotidyl transferase (TdT) activity has been reduced, substantially reduced, or eliminated. Such enzymes that are reduced or substantially reduced in terminal deoxynucleotidyl transferase activity, or in which TdT activity has been eliminated, may be obtained by mutating, for example, amino acid residues within the reverse transcriptase of interest which are in close proximity or in contact with the template-primer, for example, by introducing one or more (e.g., one, two, three, four, five, ten, twelve, fifteen, twenty, thirty, etc.) point mutations, one or more deletion mutations, and/or one or more insertion mutations. Reverse transcriptases which exhibit decreased TdT activity are described in U.S. application Ser. No. 09/808,124, filed Mar. 15, 2001 (the entire disclosure of which is incorporated herein by reference), and include reverse transcriptases with one or more alterations at amino acid positions equivalent or corresponding to Y64, M289, F309, T197 and/or Y133 of M-MLV reverse transcriptase.

Enzymes for use in the invention also include those which exhibit increased fidelity. Fidelity refers to the accuracy of polymerization, or the ability of the reverse transcriptase to discriminate correct from incorrect substrates, (e.g., nucleotides) when synthesizing nucleic acid molecules which are complementary to a template. The higher the fidelity of a reverse transcriptase, the less the reverse transcriptase misincorporates nucleotides in the growing strand during nucleic acid synthesis; that is, an increase or enhancement in fidelity results in a more faithful reverse transcriptase having decreased error rate or decreased misincorporation rate.

A reverse transcriptase having increased/enhanced/higher fidelity is defined as a polymerase having any increase in fidelity, preferably about 1.2 to about 10,000 fold, about 1.5 to about 10,000 fold, about 2 to about 5,000 fold, or about 2 to about 2000 fold (preferably greater than about 5 fold, more preferably greater than about 10 fold, still more preferably greater than about 50 fold, still more preferably greater than about 100 fold, still more preferably greater than about 500 fold and most preferably greater than about 100 fold) reduction in the number of misincorporated nucleotides during synthesis of any given nucleic acid molecule of a given length compared to the control reverse transcriptase. Reverse transcriptases which exhibit increased fidelity are described in U.S. Appl. No. 60/189,454, filed Mar. 15, 2000, U.S. application Ser. No. 09/808,124, filed Mar. 15, 2001, U.S. Appl. No. 60/056,263, filed Aug. 29, 1997, U.S. Appl. No. 60/060,131, filed Sep. 26, 1997, U.S. Appl. No. 60/085, 247, filed May 13, 1998, U.S. application Ser. No. 09/141,522, filed Aug. 27, 1998, U.S. application Ser. No. 09/677, 574, filed Aug. 3, 2000; PCT Publication No. WO 00/204022; and PCT Publication No. WO 01/68895 (the entire disclosures of each of which are incorporated herein by reference). Enzymes for use in the invention also include those in which terminal deoxynucleotidyl transferase (TdT) activity has been reduced, substantially reduced, or eliminated. Such enzymes that are reduced or substantially reduced in terminal deoxynucleotidyl transferase activity, or in which TdT activity has been eliminated, may be obtained by mutating, for example, amino acid residues within the reverse transcriptase of interest which are in close proximity or in contact with the template-primer, for example, by introducing one or more (e.g., one, two, three, four, five, ten, twelve, fifteen, twenty, thirty, etc.) point mutations, one or more deletion mutations, and/or one or more insertion mutations. Reverse transcriptases which exhibit decreased TdT activity are described in U.S. application Ser. No. 09/808,124, filed Mar. 15, 2001 (the entire disclosure of which is incorporated herein by reference), and include reverse transcriptases with one or more alterations at amino acid positions equivalent or corresponding to Y64, M289, F309, T197 and/or Y133 of M-MLV reverse transcriptase.

Enzymes for use in the invention also include those which exhibit increased fidelity. Fidelity refers to the accuracy of polymerization, or the ability of the reverse transcriptase to discriminate correct from incorrect substrates, (e.g., nucleotides) when synthesizing nucleic acid molecules which are complementary to a template. The higher the fidelity of a reverse transcriptase, the less the reverse transcriptase misincorporates nucleotides in the growing strand during nucleic acid synthesis; that is, an increase or enhancement in fidelity results in a more faithful reverse transcriptase having decreased error rate or decreased misincorporation rate.

A reverse transcriptase having increased/enhanced/higher fidelity is defined as a polymerase having any increase in fidelity, preferably about 1.2 to about 10,000 fold, about 1.5 to about 10,000 fold, about 2 to about 5,000 fold, or about 2 to about 20,000 fold (preferably greater than about 5 fold, more preferably greater than about 10 fold, still more preferably greater than about 50 fold, still more preferably greater than about 100 fold, still more preferably greater than about 500 fold and most preferably greater than about 100 fold) reduction in the number of misincorporated nucleotides during synthesis of any given nucleic acid molecule of a given length compared to the control reverse transcriptase. Reverse transcriptases which exhibit increased fidelity are described in U.S. Appl. No. 60/189,454, filed Mar. 15, 2000, U.S. application Ser. No. 09/808,124, filed Mar. 15, 2001, U.S. Appl. No. 60/056,263, filed Aug. 29, 1997, U.S. Appl. No. 60/060,131, filed Sep. 26, 1997, U.S. Appl. No. 60/085, 247, filed May 13, 1998, U.S. application Ser. No. 09/141,522, filed Aug. 27, 1998, U.S. application Ser. No. 09/677, 574, filed Aug. 3, 2000; PCT Publication No. WO 00/204022; and PCT Publication No. WO 01/68895 (the entire disclosures of each of which are incorporated herein by reference). Enzymes for use in the invention also include those in which terminal deoxynucleotidyl transferase (TdT) activity has been reduced, substantially reduced, or eliminated. Such enzymes that are reduced or substantially reduced in terminal deoxynucleotidyl transferase activity, or in which TdT activity has been eliminated, may be obtained by mutating, for example, amino acid residues within the reverse transcriptase of interest which are in close proximity or in contact with the template-primer, for example, by introducing one or more (e.g., one, two, three, four, five, ten, twelve, fifteen, twenty, thirty, etc.) point mutations, one or more deletion mutations, and/or one or more insertion mutations. Reverse transcriptases which exhibit decreased TdT activity are described in U.S. application Ser. No. 09/808,124, filed Mar. 15, 2001 (the entire disclosure of which is incorporated herein by reference), and include reverse transcriptases with one or more alterations at amino acid positions equivalent or corresponding to Y64, M289, F309, T197 and/or Y133 of M-MLV reverse transcriptase.

Enzymes for use in the invention also include those which exhibit increased fidelity. Fidelity refers to the accuracy of polymerization, or the ability of the reverse transcriptase to discriminate correct from incorrect substrates, (e.g., nucleotides) when synthesizing nucleic acid molecules which are complementary to a template. The higher the fidelity of a reverse transcriptase, the less the reverse transcriptase misincorporates nucleotides in the growing strand during nucleic acid synthesis; that is, an increase or enhancement in fidelity results in a more faithful reverse transcriptase having decreased error rate or decreased misincorporation rate.

A reverse transcriptase having increased/enhanced/higher fidelity is defined as a polymerase having any increase in fidelity, preferably about 1.2 to about 10,000 fold, about 1.5 to about 10,000 fold, about 2 to about 5,000 fold, or about 2 to about 20,000 fold (preferably greater than about 5 fold, more preferably greater than about 10 fold, still more preferably greater than about 50 fold, still more preferably greater than about 100 fold, still more preferably greater than about 500 fold and most preferably greater than about 100 fold) reduction in the number of misincorporated nucleotides during synthesis of any given nucleic acid molecule of a given length compared to the control reverse transcriptase. Reverse transcriptases which exhibit increased fidelity are described in U.S. Appl. No. 60/189,454, filed Mar. 15, 2000, U.S. application Ser. No. 09/808,124, filed Mar. 15, 2001, U.S. Appl. No. 60/056,263, filed Aug. 29, 1997, U.S. Appl. No. 60/060,131, filed Sep. 26, 1997, U.S. Appl. No. 60/095, 247, filed May 13, 1998, U.S. application Ser. No. 09/141,522, filed Aug. 27, 1998, U.S. application Ser. No. 09/677, 574, filed Aug. 3, 2000; PCT Publication No. WO 00/204022; and PCT Publication No. WO 01/68895 (the entire disclosures of each of which are incorporated herein by reference).

A variety of DNA polymerases are useful in accordance with the present invention including thermostable and mesophilic DNA polymerases. In one aspect, preferred DNA polymerases are those that have reverse transcriptase activity (template reading in the 3′ to 5′ direction) and/or DNA polymerase activity (e.g. template reading in the 5′ to 3′ direction). Such polymerases for use in the invention include, but are not limited to, pol I type and pol III type DNA polymerases. Examples of thermostable DNA polymerase for use in the invention include Thermus thermophilus (Tth) DNA polymerase, Thermus aquaticus (Taq) DNA polymerase, Thermotoga neapolitana (The) DNA polymerase, Thermotoga maritima (Tma) DNA polymerase, Thermococcus litoralis (Th or VENT™) DNA polymerase, Pyrococcusfuriosis (Pfu) DNA polymerase, Pyrococcus species GB-D (DEEPVENT™) DNA polymerase, Pyrococcus woosii (Pwo) DNA polymerase, Bacillus sterothennophilus (Bst) DNA polymerase, Bacillus caldophilus (Bca) DNA polymerase, Sulfolobus acidocaldarius (Sac) DNA polymerase, Thermoplasma acidophilum (Tac) DNA polymerase, Thermus flavus (TfllTub) DNA polymerase, Thermus ruber (Tru) DNA polymerase, Thermus brockianus (DYNAZYME™) DNA polymerase, Methanobacterium thermoautotrophicum (Mth) DNA polymerase, Mycobacterium spp. DNA polymerase (Mtb, Mlep), and mutants, variants and derivatives thereof. Mesophilic polymerases include DNA polymerase I, T5 DNA polymerase, T7 DNA polymerase, Klenow fragment DNA polymerase, DNA polymerase III, and the like.

Preferred DNA polymerases are thermostable DNA polymerases such as Taq, The, Tma, Pfu, VENT™, DEEPVENT™, Tth and mutants, variants and derivatives thereof (U.S. Pat. No. 5,436,149; U.S. Pat. No. 5,512,462; PCT Publication No. WO 92/06188; PCT Publication No. WO 92/06200; PCT Publication No. WO 96/10640; Barnes, W. M., Gene 112:29-35 (1992); Lawyer, F. C., et al., PCR Meth. AppL 2:275-287 (1993); Flaman, J.-M., et al., Nucl. Acids Res. 22(15):3259-3260 (1994)). Other DNA polymerases for use in the invention may be found in U.S. Application No. 60/318,903, filed Sep. 14, 2001, and U.S. Patent Application US 2002/0012970. For amplification of long nucleic acid molecules (e.g., nucleic acid molecules longer than about 3-5 Kb in length), at least two DNA polymerases (one substantially lacking 3′ exonuclease activity and the other having 3′ exonuclease activity) are typically used. See U.S. Pat. No. 5,436,149; U.S. Pat. No. 5,512,462; Barnes, W. M., Gene 112:29-35 (1992); PCT Publication No. WO 98/06736; and commonly owned, co-pending U.S. patent application Ser. No. 08/801,720, filed Feb. 14, 1997, the disclosures of all of which are incorporated herein in their entireties. Examples of DNA polymerases substantially lacking in 3′ exonuclease activity include, but are not limited to, Taq, Tne(exo′), Tma, Pfu(exo′), Pwo and Tth DNA polymerases, and mutants, variants and derivatives thereof. Non-limiting examples of DNA polymerases having 3′ exonuclease activity include Pfu, DEEPVENT™ and Tli/VENT™ and mutants, variants and derivatives thereof.

### Formulation of Compositions

To form the compositions of the present invention, one or more (preferably two or more) of the same or different modified nucleotides are preferably admixed in an aqueous solution such as a buffered salt solution. One or more DNA polymerases, reverse transcriptases and/or one or more nucleotides may optionally be added to make the compositions of the invention. The compositions of the invention may also comprise one or more nucleic acid templates and/or one or more primers. More preferably, the enzymes are provided at working concentrations in stable buffered salt solutions. The terms “stable” and “stability” as used herein generally mean the retention by a composition, such as an enzyme composition, of at least 70%, preferably at least 80%, and most preferably at least 90%, of the original enzymatic activity (in units) after the enzyme or composition containing the enzyme has been stored for about one week at a temperature of about 4° C., about two to six months at a temperature of about −20° C., and about six months or longer at a temperature of about −80° C. As used herein, the term “working concentration” means the concentration of an enzyme that is at or near the optimal concentration used in a solution to perform a particular function (such as reverse transcription of nucleic acids).

The water used in forming the composition of the present invention is preferably distilled, deionized and sterile filtered (through a 0.1-0.2 micrometer filter), and is free of contamination by DNase and RNase enzymes. Such water is available commercially, for example from Sigma Chemical Company (Saint Louis, Mo.), or may be made as needed according to methods well known to those skilled in the art.

In addition to the enzyme components, the present compositions preferably comprise one or more buffers and cofactors necessary for synthesis of nucleic acid molecules comprising one or more modified nucleotides or labeled nucleic acid molecules of the invention. Particularly preferred buffers for use in forming the present compositions are the acetate, sulfate, hydrochloride, phosphate or free acid forms of Tris-(hydroxymethyl)aminomethane (TRIS), although alternative buffers of the same approximate ionic strength and pKa as TRIS may be used with equivalent results. In addition to the buffer salts, cofactor salts such as those of potassium (preferably potassium chloride or potassium acetate) and magnesium (preferably magnesium chloride or magnesium acetate) are included in the compositions. Addition of one or more carbohydrates and/or sugars to the compositions and/or synthesis reaction mixtures may also be advantageous, to support enhanced stability of the compositions and/or reaction mixtures upon storage. Preferred such carbohydrates or sugars for inclusion in the compositions and/or synthesis reaction mixtures of the invention include, but are not limited to, sucrose, trehalose, and the like. Furthermore, such carbohydrates and/or sugars may be added to the storage buffers for the enzymes used in the production of the enzyme compositions and kits of the invention. Such carbohydrates and/or sugars are commercially available from a number of sources, including Sigma (St. Louis, Mo.).

It is often preferable to first dissolve the buffer salts, cofactor salts and carbohydrates or sugars at working concentrations in water and to adjust the pH of the solution prior to addition of the enzymes. In this way, pH-sensitive enzymes will be less subject to acid- or alkaline-mediated inactivation during formulation of the present compositions.

Concentrations of the RTs in the compositions of the invention may vary depending on the type of reverse transcriptase used. For example, AMV RTs, MAV RTs, RSV RTs and RAV RTs are preferably added at a working concentration in the solution of about 100 to about 5000 units per milliliter, about 125 to about 4000 units per milliliter, about 150 to about 3000 units per milliliter, about 200 to about 2500 units per milliliter, about 225 to about 2000 units per milliliter, and most preferably at a working concentration of about 250 to about 1000 units per milliliter. The enzymes in the thermophilic DNA polymerase group and mutants, variants and derivatives thereof are preferably added at a working concentration in the solution of about 100 to about 1000 units per milliliter, about 125 to about 750 units per milliliter, about 150 to about 700 units per milliliter, about 200 to about 650 units per milliliter, about 225 to about 550 units per milliliter, and most preferably at a working concentration of about 250 to about 500 units per milliliter. The enzymes may be added to the solution in any order, or combination, and may be added simultaneously.

The compositions of the invention may further comprise one or more nucleotides, which are preferably deoxynucleotide triphosphates (dNTPs). The dNTP components of the present compositions serve as the “building blocks” for newly synthesized nucleic acids, being incorporated therein by the action of the polymerases or reverse transcriptases.

Production of Nucleic Acid or cDNA Molecules

In accordance with the invention, nucleic acid or cDNA molecules (single-stranded or double-stranded) may be prepared from a variety of nucleic acid template molecules. Preferred templates for use in the present invention include single-stranded or double-stranded DNA and RNA molecules, as well as double-stranded DNA:RNA hybrids. More preferred templates include messenger RNA (mRNA), transfer RNA (tRNA) and ribosomal RNA (rRNA) molecules, although mRNA molecules are the preferred templates according to the invention.

Preferably the nucleic acid templates may be obtained from natural sources, such as a variety of cells, tissues, organs or organisms. Cells that may be used as sources of nucleic acid molecules may be prokaryotic (bacterial cells, including but not limited to those of species of the genera Escherichia, Bacillus, Serratia, Salmonella, Staphylococcus, Streptococcus, Clostridium, Chlamydia, Neisseria, Treponema, Mycoplasma, Borrelia, Legionella, Pseudomonas, Mycobacterium, Helicobacter, Erwinia, Agrobacterium, Rhizobium, Xanthomonas and Streptomyces) or eukaryotic (including fungi (especially yeasts), plants, protozoan and other parasites, and animals including insects (particularly Drosophila spp. cells), nematodes (particularly Caenorhabditis elegans cells), and mammals (particularly human cells)).

Mammalian somatic cells that may be used as sources of nucleic acids include blood cells (reticulocytes and leukocytes), endothelial cells, epithelial cells, neuronal cells (from the central or peripheral nervous systems), muscle cells (including myocytes and myoblasts from skeletal, smooth or cardiac muscle), connective tissue cells (including fibroblasts, adipocytes, chondrocytes, chondroblasts, osteocytes and osteoblasts) and other stromal cells (e.g., macrophages, dendritic cells, Schwann cells). Mammalian germ cells (spermatocytes and oocytes) may also be used as sources of nucleic acids for use in the invention, as may the progenitors, precursors and stem cells that give rise to the above somatic and germ cells. Also suitable for use as nucleic acid sources are mammalian tissues or organs such as those derived from brain, kidney, liver, pancreas, blood, bone marrow, muscle, nervous, skin, genitourinary, circulatory, lymphoid, gastrointestinal and connective tissue sources, as well as those derived from a mammalian (including human) embryo or fetus.

Any of the above prokaryotic or eukaryotic cells, tissues and organs may be normal, diseased, transformed, established, progenitors, precursors, fetal or embryonic. Diseased cells may, for example, include those involved in infectious diseases (caused by bacteria, fungi or yeast, viruses (including AIDS, HIV, HTLV, herpes, hepatitis and the like) or parasites, in genetic or biochemical pathologies (e.g., cystic fibrosis, hemophilia, Alzheimer's disease, muscular dystrophy or multiple sclerosis) or in cancerous processes. Transformed or established animal cell lines may include, for example, COS cells, CHO cells, VERO cells, BHK cells, HeLa cells, HepG2 cells, K562 cells, 293 cells, L929 cells, F9 cells, and the like. Other cells, cell lines, tissues, organs and organisms suitable as sources of nucleic acids for use in the present invention will be apparent to one of ordinary skill in the art.

Once the starting cells, tissues, organs or other samples are obtained, nucleic acid templates (such as mRNA) may be isolated there from by methods that are well-known in the art (See, e.g., Maniatis, T., et al., Cell 15:687-701 (1978); Okayama, H., and Berg, P., Mol. Cell. Biol. 2:161-170 (1982); Gubler, U., Hoffman, B. J., Gene 25:263-269 (1983) (PCT Publication No. WO 98/08981; PCT Publication No. WO 98/51699; and PCT Publication No. WO 00/52191). The nucleic acid molecules thus isolated may then be used to prepare cDNA molecules and cDNA libraries in accordance with the present invention.

In the practice of the invention, nucleic acid molecules (which may be labeled) are produced by mixing one or more nucleic acid molecules obtained as described above, which is preferably one or more mRNA molecules such as a population of mRNA molecules, with one or more (preferably two or more) of the same or different modified nucleotides and one or more polypeptides having reverse transcriptase activity and/or DNA polymerase activity, under conditions favoring the reverse transcription or synthesis of the nucleic acid molecule by the action of the enzymes or the compositions to form one or more nucleic acid molecules (single-stranded or double-stranded which may include cDNA) having one or more modified nucleotides (which may be the same or different) incorporated therein, The newly synthesized molecules may then be labeled by coupling a detectable label to one or more of the modified nucleotides incorporated in the synthesized nucleic acid molecule. Thus, the method of the invention comprises (a) mixing one or more nucleic acid templates (preferably one or more RNA or mRNA templates, such as a population of mRNA molecules) with one or more reverse transcriptases and/or DNA polymerases and one or more and preferably two or more modified nucleotides, (b) incubating the mixture under conditions sufficient to make one or more nucleic acid molecules complementary to all or a portion of the one or more templates. The invention may be used in conjunction with methods of cDNA synthesis such as those described in the Examples below, or others that are well-known in the art (see, e.g., Gubler, U., and Hoffman, B. J., Gene 25:263-269 (1983); Krug, M. S., and Berger, S. L., Meth. Enzymol. 152:316-325 (1987); Sambrook, J., et al., Molecular Cloning: A Laboratory Manual, 2nd ed., Cold Spring Harbor, N.Y.: Cold Spring Harbor Laboratory Press (1989), pp. 8.60-8.63; PCT Publication No. WO 99/15702; PCT Publication No. WO 98/47912; PCT Publication WO 98/08981; PCT Publication No. WO 98/51699; PCT Publication No. WO 00/52191; and PCT Publication No. WO 98/51699), to produce cDNA molecules or libraries.

In other aspects, the invention may be used in methods for amplifying nucleic acid molecules. Nucleic acid amplification methods according to this aspect of the invention may be one-step (e.g., one-step RT-PCR) or two-step (e.g., two-step RT-PCR) reactions. According to the invention, one-step RT-PCR type reactions may be accomplished in one tube thereby lowering the possibility of contamination. Such one-step reactions comprise (a) mixing a nucleic acid template (e.g., mRNA) with one or more polypeptides having reverse transcriptases activity and with one or more DNA polymerases and one or more (preferably two or more) modified nucleotides (b) incubating the mixture under conditions sufficient to amplify one or more nucleic acid molecule complementary to all or a portion of the template. Such conditions for amplification may include the use of one or more nucleotides, one or more primers, and one or more suitable buffers. Alternatively, amplification may be accomplished by mixing one or more templates with one or more polypeptides having reverse transcriptase activity. Incubating such a reaction mixture under appropriate conditions allows amplification of a nucleic acid molecule having one or more (preferably two or more) modified nucleotides incorporated therein and which is complementary to all or a portion of the template. Such amplification may be accomplished by the reverse transcriptase activity alone or in combination with a DNA polymerase. Two-step RT-PCR reactions may be accomplished in two separate steps. Such a method comprises (a) mixing a nucleic acid template (e.g., mRNA) with one or more reverse transcriptases, (b) incubating the mixture under conditions sufficient to make a first nucleic acid molecule (e.g., a DNA molecule) complementary to all or a portion of the template, (c) mixing the first nucleic acid molecule with one or more DNA polymerases and (d) incubating the mixture of step (c) under conditions sufficient to amplify the first nucleic acid molecule. Conditions in step (b) and/or step (d) may include the use of one or more (preferably two or more) modified nucleotides, one or more nucleotides, one or more primers and one or more suitable buffers. Such amplification allows production of nuclei acid molecules comprising one or more (preferably two or more) modified nucleotides which may be the same or different. For amplification of long nucleic acid molecules (i.e., greater than about 3-5 Kb in length), a combination of DNA polymerases may be used, such as one DNA polymerase having 3′ exonuclease activity and another DNA polymerase being substantially reduced or lacking in 3′exonuclease activity.

Amplification methods which may be used in accordance with the present invention include PCR (U.S. Pat. Nos. 4,683,195 and 4,683,202), Strand Displacement Amplification (SDA; U.S. Pat. No. 5,455,166; EP 0 684 315), and Nucleic Acid Sequence-Based Amplification (NASBA; U.S. Pat. No. 5,409,818; EP 0 329 822).

Labeling cDNA Molecules

In accordance with the invention, methods of labeling nucleic acid molecules or cDNA molecules (single-stranded or double-stranded) are provided. First, a nucleic acid template is reversed transcribed incorporating one or more (preferably two or more) of the same or different modified nucleotides into the synthesized nucleic acid molecule. In a preferred embodiment, the nucleic acid template is RNA, more preferably mRNA. Also, in a preferred embodiment, the modified nucleotides are selected from amino-modified dUTP and dATP. If the nucleic acid template is RNA, the RNA is preferably subsequently degraded by base hydrolysis, and the reaction is neutralized with acid. The amino-modified cDNA is then preferably purified to remove unincorporated nucleotides and primers. In a second step, the synthesized nucleic acid molecule is coupled with one or more detectable labels (which may be the same or different). In a preferred embodiment, the detectable label is a fluorescent dye. In a more preferred embodiment the cDNA produced in the first step is coupled with the monoreactive succinimide ester derivative of a fluorescent dye. In a more preferred embodiment, the fluorescent dye is Cy3 or Cy5. The resulting fluorescently labeled cDNA may be purified with a Snap™ spin column, to remove any unreacted dye. The purified fluorescently labeled cDNA may then be used for hybridization to one or more nucleic acid molecules (preferably single stranded nucleic acid molecules) or to arrays. In preferred embodiments of the present invention, the detectable label is a fluorescent dye available in the N-hydroxysuccinimide reactive form. Such dyes include, but are not limited to, Alexa, Oyster, Fluorescene, Texas Red, FITC, Rhodamin, Cy3, and Cy5.

### Kits

In another embodiment, the present invention may be assembled into kits for use in reverse transcription, synthesis or amplification of a nucleic acid molecule. Kits according to this aspect of the invention comprise a carrier means, such as a box, carton, tube or the like, having in close confinement therein one or more container means, such as vials, tubes, ampules, bottles and the like, comprising one or more (preferably two or more) modified nucleotides which may be the same or different and may be in the same or separate containers. The kits of the invention may also comprise (in the same or separate containers), one or more polypeptides of the invention having reverse transcriptase activity, one or more DNA polymerases, a suitable buffer, one or more nucleotides, one or more labels, one or more solid supports, one or more arrays, one or more labeled nucleotides (which may include fluorescent nucleotides which may be the same or different) and/or one or more primers.

In a specific aspect of the invention, the reverse transcription, synthesis and amplification kits may comprise one or more components (in mixtures or separately) including one or more polypeptides having reverse transcriptase activity of the invention, one or more nucleotides needed for synthesis of a nucleic acid molecule, one or more modified nucleotides, and/or one or more primers (e.g., oligo(dT) for reverse transcription). Such kits may further comprise one or more DNA polymerases. Preferred polypeptides having reverse transcriptase activity, DNA polymerases, nucleotides, modified nucleotides, solid supports, arrays, primers and other components suitable for use in the kits of the invention include those described herein. The kits encompassed by this aspect of the present invention may further comprise additional reagents and compounds necessary for carrying out standard nucleic acid reverse transcription, synthesis, or amplification protocols and those described herein. Such kits may also comprise instructions for carrying out the method and protocols in accordance with the invention.

In a preferred embodiment of the present invention, the kit will contain reagents for labeling cDNA molecules and purifying cDNA molecules labeled with a fluorescent dye. The kit may contain one or more components selected from SuperScript™, SuperScript II, SuperScript™ III, a buffer, anchored oligo (dT)₂₀ (which may be anchored to a solid support)and random primers (which may be random primers), coupling buffer, and S.N.A.P.™ columns and buffers for a sample cleanup. The kit may also include reagents for dissolving dye esters, reagents for reaction quenching, controls and a detailed instruction manual.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLE 1

### cDNA Synthesis with Mixture of AA-dUTP and AH-dATP

### Total HeLa RNA

We prepared two first-strand cDNA synthesis reactions using total

RNA as starting material: one to measure incorporation of aminoallyl-dUTP (AA-dUTP) alone, and one to measure incorporation of AA-dUTP plus aminohexyl-dATP (AH-dATP). Each reaction was set up using 10 μg of total HeLa RNA primed with 5 μg of oligo(dT)₂₀-VN. (Note: V stands for either dG, dA, or dC; N stands for either dG, dA, dT, or dC). This mixture was heated to 70° C. for 5 minutes, and then placed on ice for at least 2 minutes.

To the reaction measuring AA-dUTP incorporation, we added 50 MM Tris-HCl (pH 8.3), 75 mM KCl, 3 mM MgCl₂, 10 mM DTT, 1 μCi ³²P-α-dCTP, 0.5 mM dGTP, 0.5 mM dCTP, 0.5 mM dATP, 0.2 mM dTTP, 0.3 mM AA-dUTP and 40 Units of RNaseOUT™.

To the reaction measuring AA-dUTP plus AH-dATP incorporation, we added 50 mM Tris-HCl (pH 8.3), 75 mM KCl, 3 mM MgCl₂, 10 mM DTT, 1 μCi ³²P-α-dCTP, 0.5 mM dGTP, 0.5 MM dCTP, 0.35 mM dATP, 0.35 mM dTTP, 0.15 mM AA-dUTP, 0.15 mM AH-dATP, and 40 Units of RNaseOUT.

SuperScript™ III Reverse Transcriptase (400 Units) was added to each reaction and the reaction volume was brought to 30 μl. Each reaction was incubated at 46° C. for 2 hours. The reactions were stopped by adding 10 μl of 0.5 M EDTA. Five microliters of each reaction were spotted onto a glass fiber (GF/C) filter, and the first-strand cDNA yield was calculated by TCA-precipitated ³²P counts, as described below.

### In Vitro Transcript RNA

We prepared two first-strand cDNA synthesis reactions using in vitro transcript RNA as starting material: one to measure AA-dUTP incorporation and one to measure AA-dUTP plus AH-dATP incorporation. Each reaction was set up using 1 μg of RNA ladder (Invitrogen) primed with 5 μg of oligo(dT)₂₀-VN. This mixture was heated to 70° C. for 5 minutes, and then placed on ice for at least 2 min.

To the reaction measuring AA-dUTP incorporation, we added 50 mM Tris-HCl (pH 8.3), 75 mM KCl, 3 mM MgCl₂, 10 mM DTT, 1 μCi ³²P-α-dCTP, 0.5 MM dGTP, 0.5 mM dCTP, 0.5 mM dATP, 0.2 mM dTTP, 0.3 mM AA-dUTP and 40 Units of RNaseOUT™.

To the reaction measuring AA-dUTP plus AH-dATP incorporation, we added 50 mM Tris-HCl (pH 8.3),75 mM KCl, 3 mM MgCl₂, 10 mM DTT, 1 μCi ³²P-α-dCTP, 0.5 mM dGTP, 0.5 mM dCTP, 0.35 mM dATP, 0.35 mM dTTP, 0.15 mM AA-dUTP, 0.15 mM AH-dATP, and 40 Units of RNaseOUT™.

SuperScript™ III Reverse Transcriptase (400 Units) was added to each reaction and the reaction volume was brought to 30 μl. Each reaction was incubated at 46° C. for 2 hours. The reactions were stopped by adding 10 μg of 0.5 M EDTA. Five microliters of each reaction were spotted onto a glass fiber (GF/C) filter, and the first-strand cDNA yield was calculated by TCA-precipitated ³²P counts, as described below.

TCA Precipitation and First-Strand cDNA Synthesis Calculation

To calculate the specific activity (SA) of ³²P, 2 μl of each sample were spotted onto GF/C filter and the cpms were counted without TCA wash:

$$SA ( cpm / pmole dCTP ) = \frac{cpm of 2 microliters from unwashed sample}{15000 pmole dCTP} \times 20$$

The GF/C filters containing 5 μl of reaction mixture were washed with ice-cold 10% (w/v) TCA, 1% sodium pyrophosphate (NaPPi) solution for 5 minutes once and with 5% TCA solution for 5 minutes twice at room temperature. After the washes, the filters were washed with 95% ethanol for 5 minutes and then dried under a heat lamp. The washed filters were counted in a standard scintillation cocktail (Ecolite, ICN, Cat. no. 882475) to determine the amount of ³²P that was incorporated. The equation used for calculating first-strand synthesis yield is:

$$Amount of cDNA ( pmol ) = \frac{cpm of washed sample}{SA} \times 8 \times 4 ( pmole dNTP / pmole dCTP )$$

The cDNA synthesis yields are shown below (the average of two reactions):

| | cDNA yield (pmol) | |
|---|---|---|
| Amino-modified nucleotide(s) | 10 μg total HeLa RNA | 1 μg RNA ladder |
| AA-dUTP | 4118 | 1199 |
| AA-dUTP + AH-dATP | 4543 | 1190 |

### EXAMPLE 2

### Cy5 Coupling into cDNA Synthesized with AA-dUTP or AH-dATP

First Strand cDNA Synthesis Using Modified Nucleotide

We prepared two first-strand cDNA synthesis reactions using total HeLa RNA as starting material: one to measure incorporation of AA-dUTP and one to measure incorporation of AH-dATP. Each reaction was set up using 10 μg of total HeLa RNA primed with 5 μg of oligo(dT)₂₀-VN. This mixture was heated to 70° C. for 5 minutes, and then placed on ice for at least 2 minutes.

To the reaction measuring AA-dUTP incorporation, we added a buffer containing 50 mM Tris-HCl (pH 8.3), 75 mM KCl, 3 mM MgCl₂, 10 mM DTT, 0.5 mM dGTP, 0.5 mM dCTP, 0.5 mM dATP, 0.2 mM dTTP, 0.3 m1v1 AA-dUTP and 40 Units of RNaseOUT™.

To the reaction measuring AH-dATP incorporation, we added a buffer containing 50 mM Tris-HCl (pH 8.3), 75 mM KCl, 3 mM MgCl₂, 10 mM DTT, 0.5 mM dGTP, 0.5 min dCTP, 0.5 min dTTP, 0.2 min dATP, 0.3 mM AH-dATP and 40 Units of RNaseOUT™.

SuperScript™ II Reverse Transcriptase (400 Units) was added to each reaction and the reaction volume was brought to 30 μl. Each reaction was incubated at 42° C. for 2 hours. The reactions were stopped by adding 15 μl of NaOH, then mixed briefly and incubated at 70° C. for 10 min. Fifteen microliters of I N HCl were added to neutralize the pH.

Purification of cDNA and Fluorescent Dye Coupling

We added 20 μl of 3 M NaAc, pH 5.2, and 500 μl of Loading Buffer (2.25 M guanidiniun HCl in 70% isopropanol) to each reaction mixture. Each mixture was vortexed briefly, loaded onto a S.N.A.P.™ column, and centrifuged at 14,000×g for 1 minute. We discarded the flowthrough, added 700 μl of wash buffer (100 mm NaCl in 75% ethanol), and centrifuged at 14,000×g for 1 minute. We discarded the flowthrough, and repeated this wash step one more time. Then we centrifuged the column at 14,000×g for 1 minute more to spin down any residual buffer.

We transferred each S.N.A.P.™ column to a new 1.5-ml tube and added 50 μl of dH₂O. We incubated this at room temperature for 1 minute, centrifuged at 14,000×g for 1 minute, and collected the flowthrough. We repeated this elution step one more time and collected the flowthrough. The total volume in each collection tube was about 100 μl.

We added 10 μl of 3M NaAc, pH 5.2, and 2 μl of 20 mg/ml glycogen to each tube, mixed briefly, and then added 300 μl of 100% ethanol. We stored the tubes at −20° C. for at least 30 minutes. We centrifuged each tube at 14,000×g for 10 minutes and carefully discarded the supernatant. We then added 250 μl of 75% ethanol to each tube, mixed gently, and centrifuged at 14,000×g for 2 minutes. We carefully discarded the supernatant and air-dried each pellet for 10 minutes. We then resuspended each pellet in 5 μl of 2× Coupling Buffer (0.1 M Sodium tetraborate, pH 8.5).

We resuspended a pack of Monofunctional Cy5Tm dye (Amersham, cat #PA25001) in 45 μl of DMSO, and added 5 pg of the Cy5™ dye to each cDNA sample in the 2× Coupling Buffer. We mixed briefly and stored the reactions at room temperature in the dark for 1 hour. We then added 5 μl of 4M hydroxylamine and stored the reactions at room temperature in the dark for 15 minutes.

Cy5-Labeled cDNA Purification

To purify the dye-labeled cDNA, we added 20 μl of 3M NaAc, pH 5.2, and 500 μl of Loading Buffer to each coupling reaction. We mixed briefly and loaded each labeled cDNA mixture onto a S.N.A.P.™ column. We centrifuged at 14,000×g for 1 minute and discarded the flowthrough. Then we added 700 μl of Wash Buffer, centrifuged at 14,000×g for 1 minute, and discarded the flowthrough. We repeated this wash and spin step one more time, and then performed another centrifugation at 14,000×g for 1 minute to spin down any residual buffer. We transferred each S.N.A.P.™ column to a new 1.5-ml amber tube and added 50 μl of dH₂O. We incubated the columns at room temperature for 1 minute and then centrifuged at 14,000×g for 1 minute, collecting the flowthrough.

### Determining the Amount of Coupled Cy5 and the Ratio of Nucleotide:Dye

The incorporation of Cy5 into the amino-modified cDNA was quantified with UV visible spectroscopy scanning. Cy5 has an absorption maximum at 650 nm. Each tube of column-purified labeled cDNA was scanned at 240-800 nm. The amount of coupled Cy5 and the ratio of nucleotide:dye were calculated as follows:

$$Cy 5 ( pmol ) = \frac{{OD}_{650} - {OD}_{750}}{0.25} \times 50$$

$$cDNA ( pmol ) = \frac{{OD}_{260} - {OD}_{520}}{0.33} \times 40 \times 50$$

$$Ratio = \frac{cDNA ( pmol )}{Cy 5 ( pmol )}$$

cDNA labeled with Cy5 from 10 μg of total HeLa RNA:

| | cDNA (pmol) | Cy5 (pmol) | Ratio |
|---|---|---|---|
| AH-dATP | 2600 | 82 | 32 |
| AA-dUTP | 3700 | 50 | 74 |

### EXAMPLE 3

### Cy3 Coupling to cDNA Synthesized with Mixture of AA-dUTP or AH-dATP

First Strand cDNA Synthesis Using Modified Nucleotide

We prepared two first-strand cDNA synthesis reactions using total HeLa RNA as starting material: one to measure incorporation of AA-dUTP and one to measure incorporation of AH-dATP. Each reaction was set up using 10 μg of total HeLa RNA primed with 5 μg of oligo(dT)₂₀-VN. This mixture was heated to 70° C. for 5 minutes, and then placed on ice for at least 2 minutes.

To the reaction measuring AA-dUTP incorporation, we added a buffer containing 50 mM Tris-HCl (pH 9.3),75 mM KCl, 3 mM MgCl₂, 10 mM DTT, 0.5 mM dGTP, 0.5 mM dCTP, 0.5 mM dATP, 0.2 min dTTP, 0.3 mM AA-dUTP and 40 Units of RNaseOUT™.

To the reaction measuring AH-dATP incorporation, we added a buffer containing 50 mM Tris-HCl (pH 8.3),75 mM KCl, 3 mM MgCl₂, 10 mM DTT, 0.5 mM dGTP, 0.5 mM dCTP. 0.5 mM dTTP, 0.2 mM dATP, 0.3 mM AH-dATP and 40 Units of RNaseOUT™

SuperScript™ III Reverse Transcriptase (400 Units) was added to each reaction and the reaction volume was brought to 30 μl. Each reaction was incubated at 46° C. for 2 hours. The reactions were stopped by adding 15 μl of NaOH, then mixed briefly and incubated at 70° C. for 10 min. Fifteen microliters of 1 N HCl were added to neutralize the pH.

Purification of cDNA and Fluorescent Dye Coupling

We added 20 μl of 3 M NaAc, pH 5.2, and 500 μl of Loading Buffer (2.25 M guanidiniun HCl in 70% isopropanol) to each reaction mixture. Each mixture was vortexed briefly, loaded onto a S.N.A.P.™ column, and centrifuged at 14,000×g for 1 minute. We discarded the flowthrough, added 700 μl of wash buffer (100 mM NaCl in 75% ethanol), and centrifuged at 14,000×g for 1 minute. We discarded the flowthrough, and repeated this wash step one more time. Then we centrifuged the column at 14,000×g for 1 minute more to spin down any residual buffer.

We transferred each S.N.A.P.™ column to a new 1.5-ml tube and added 50 μl of dH₂O. We incubated this at room temperature for 1 minute, centrifuged at 14,000×g for 1 minute, and collected the flowthrough. We repeated this elution step one more time and collected the flowthrough. The total volume in each collection tube was about 100

We added 10 μl of 3M NaAc, pH 5.2, and 2 μl of 20 mg/ml glycogen to each tube, mixed briefly, and then added 300 μl of 100% ethanol. We stored the tubes at −20° C. for at least 30 minutes. We centrifuged each tube at 14,000×g for 10 minutes and carefully discarded the supernatant. We then added 250 μl of 75% ethanol to each tube, mixed gently, and centrifuged at 14,000×g for 2 minutes. We carefully discarded the supernatant and air-dried each pellet for 10 minutes. We then resuspended each pellet in 5 μl of 2× Coupling Buffer (0.1 M Sodium tetraborate, pH 8.5).

We resuspended a pack of Monofunctional Cy3 dye (Amersham, cat #PA23001) in 45 μl of DMSO, and added 5 μl of the Cy3 dye to each cDNA sample in the 2× Coupling Buffer. We mixed briefly and stored the reactions at room temperature in the dark for 1 hour. We then added 5 μl of 4M hydroxylamine and stored the reactions at room temperature in the dark for 15 minutes.

Cy3-Labeled cDNA Purification

To purify the dye-labeled cDNA, we added 20 μl of 3M NaAc, pH 5.2, and 500 μl of Loading Buffer to each coupling reaction. We mixed briefly and loaded each labeled cDNA mixture onto a S.N.A.P.™ column. We centrifuged at 14,000×g for 1 minute and discarded the flowthrough. Then we added 700 p.1 of Wash Buffer, centrifuged at 14,000×g for 1 minute, and discarded the flowthrough. We repeated this wash and spin step one more time, and then performed another centrifugation at 14,000×g for 1 minute to spin down any residual buffer. We transferred each S.N.A.P.™ column to a new 1.5-ml amber tube and added 50 μl of dH₂O. We incubated the columns at room temperature for 1 minute and then centrifuged at 14,000×g for 1 minute, collecting the flowthrough.

### Determining the Amount of Coupled Cy3 and the Ratio of Nucleotide:Dye

The incorporation of Cy3 into the amino-modified cDNA was quantified with UV visible spectroscopy scanning. Cy3 has an absorption maximum at 550 nm. Each tube of column-purified labeled cDNA was scanned at 240-800 nm. The amount of coupled Cy3 and the ratio of nucleotide:dye were calculated as follows:

$$Cy 3 ( pmol ) = \frac{{OD}_{550} - {OD}_{650}}{0.15} \times 50$$

$$cDNA ( pmol ) = \frac{{OD}_{260} - {OD}_{520}}{0.33} \times 40 \times 50$$

$$Ratio = \frac{cDNA ( pmol )}{Cy 3 ( pmol )}$$

cDNA labeled with Cy3 from 10 μg of total HeLa RNA:

| | cDNA (pmol) | Cy3 (pmol) | Ratio |
|---|---|---|---|
| AA-dUTP | 1954 | 45 | 44 |
| AA-dUTP + AH-dATP | 2147 | 59 | 37 |

### EXAMPLE 4

### Optimal Temperature of cDNA Synthesis

First-Strand cDNA Synthesis Using Different Temperatures

We prepared five first-strand cDNA synthesis reactions using total HeLa RNA as starting material. Each reaction was set up using 10 μg of total HeLa RNA primed with 5 μg of oligo(dT)₂₀-VN. This mixture was heated to 70° C. for 5 minutes, and then placed on ice for at least 2 minutes.

To each reaction we added a reaction buffer of 50 mM Tris-HCl (pH 8.3), 75 mM KCl, 3 mM MgCl₂, 10 mM DTT, 1 μCi ³²P-α-dCTP, 0.5 mM dGTP, 0.5 mM dCTP, 0.35 mM dATP, 0.35 mM dTTP, 0.15 mM AA-dUTP, 0. 15 mM AH-dATP and 40 Units of RNaseOUT.

SuperScript™ III Reverse Transcriptase (400 Units) was added to each reaction and the reaction volume was brought to 30 μl. Each reaction was then incubated at 42° C., 44° C., 46° C., 48° C., or 50° C. for 2 hours. The reactions were stopped by adding 10 μl of 0.5 M EDTA. Five microliters of each reaction were spotted onto a glass fiber (GF/C) filter, and the first-strand cDNA yield was calculated by TCA-precipitated ³²P counts, as described below.

TCA Precipitation and First-Strand cDNA Synthesis Calculation

To calculate the specific activity (SA) of ³²P, 2 μl of each sample were spotted onto GF/C filter and the cpms were counted without TCA wash:

$$SA ( cpm / pmole dCTP ) = \frac{cpm of 2 microliters from unwashed sample}{15000 pmole dCTP} \times 20$$

The GF/C filters containing 5 μl of reaction mixture were washed with ice-cold 10% (w/v) TCA, 1% sodium pyrophosphate (NaPPi) solution for 5 minutes once and with 5% TCA solution for 5 minutes twice at room temperature. After the washes, the filters were washed with 95% ethanol for 5 minutes and then dried under a heat lamp. The washed filters were counted in a standard scintillation cocktail (Ecolite, ICN, Cat. no. 882475) to determine the amount of ³²P in the reaction, as well as the amount of ³²P that was incorporated. The equation used for calculating first-strand synthesis yield is:

$$A mount of cDNA ( pmol ) = \frac{cpm of washed sample}{SA} \times 8 \times 4 ( pmole dNTP / pmole dCTP )$$

The cDNA yield for each reaction is shown in the table below:

| | cDNA yield (pmol) |
|---|---|
| 42° C. | 1583 |
| 44° C. | 1435 |
| 46° C. | 1734 |
| 48° C. | 1402 |
| 50° C. | 911 |

### EXAMPLE 5

### Optimal Amount of SuperScript™ III RT for cDNA Synthesis

First-Strand cDNA Synthesis Using Different Amounts of SuperScript™ III RT

We prepared three first-strand cDNA synthesis reactions using total HeLa RNA as starting material. Each reaction was set up using 10 μg of total HeLa RNA primed with 5 μg of oligo(dT)₂₀-VN. This mixture was heated to 70° C. for 5 minutes, and then placed on ice for at least 2 minutes.

To each reaction we added a reaction buffer of 50 mM Tris-HCl (pH 83), 75 mM KCl, 3 mM MgCl₂, 10 mM DTT, 1 μCi ³²P-α-dCTP, 0.5 mM dGTP, 0.5 mM dCTP, 0.35 mM dATP, 0.35 mM dTTP, 0.15 mM AA-dUTP, 0. 15 mM AH-dATP and 40 Units of RNaseOUT.

To each reaction mixture was added 400, 800 or 1,000 Units of SuperScript™ III RT. The total volume of each reaction was 30 μl. Then each reaction was incubated at 46° C. At 1 hour, 2 hours, 4 hours, and 6 hours, 5 μl of each reaction were spotted onto a glass fiber (GF/C) filter. The first-strand cDNA yield was calculated by TCA-precipitated ³²P counts, as described below.

TCA Precipitation and First-Strand cDNA Synthesis Calculation

To calculate the specific activity (SA) of ³²P, 2 μl of each reaction were spotted onto GF/C filter and the cams were counted without TCA wash:

$$SA ( cpm / pmole dCTP ) = \frac{cpm of 2 microliters from unwashed sample}{15000 pmole dCTP} \times 20$$

The GF/C filters containing 5 μl of reaction mixture were washed with ice-cold 10% (w/v) TCA, 1% sodium pyrophosphate (NaPPi) solution for 5 minutes once and with 5% TCA solution for 5 minutes twice at room temperature. After the washes, the filters were washed with 95% ethanol for 5 minutes and then dried under a heat lamp. The washed filters were counted in a standard scintillation cocktail to determine the amount of ³²P in the reaction, as well as the amount of ³²P that was incorporated. The equation used for calculating first-strand synthesis yield is:

$$Amount of cDNA ( pmol ) = \frac{cpm of washed sample}{SA} \times 8 \times 4 ( pmole dNTP / pmole dCTP )$$

The cDNA yield for each reaction at the different time points is shown in the table below:

| SuperScript ™ | 1 hr | 2 hrs | 4 hrs | 6 hrs |
|---|---|---|---|---|
| III RT (Units) | (pmol) | (pmol) | (pmol) | (pmol) |
| 400 | 1217 | 1741 | 2948 | 3213 |
| 800 | 1589 | 2814 | 3740 | 3872 |
| 1000 | 1932 | 2694 | 3889 | 4406 |

### EXAMPLE 6

### Optimal Amount of SuperScript™ II RT for cDNA Synthesis

First-Strand cDNA Synthesis Using Different Amounts of SuperScript™ II RT

We prepared three first-strand cDNA synthesis reactions using total HeLa RNA as starting material. Each reaction was set up using 10 μg of total HeLa RNA primed with 5 μg of oligo(dT)₂₀-VN. This mixture was heated to 70° C. for 5 minutes, and then placed on ice for at least 2 minutes.

To each reaction we added a reaction buffer of 50 mM Tris-HCl (pH 8.3), 75 mM KCl, 3 mM MgCl₂, 10 mM DTT, 1 μCi ³²P-α-dCTP, 0.5 mM dGTP, 0.5 mM dCTP, 0.35 mM dATP, 0.35 mM dTTP, 0. 15 mM AA-dUTP, 0.15 mM AH-dATP and 40 Units of RNaseOUT.

To each reaction mixture was added 400, 800 or 1,000 Units of SuperScript™ II RT. The total volume of each reaction was 30 μl. Then the reaction was incubated at 46° C. At 1 hour, 2 hours, 4 hours, and 6 hours, 5 μl of each reaction were spotted onto a glass fiber (GF/C) filter. The first-strand cDNA yield was calculated by TCA-precipitated ³²P counts, as described below.

TCA Precipitation and First-Strand cDNA Synthesis Calculation

To calculate the specific activity (SA) of ³²P, 2 μl of each reaction were spotted onto GF/C filter and the cams were counted without TCA wash:

$$SA ( cpm / pmole dCTP ) = \frac{cpm of 2 microliters from unwashed sample}{15000 pmole dCTP} \times 20$$

The GF/C filters containing 5 μl of reaction mixture were washed with ice-cold 10% (w/v) TCA, 1% sodium pyrophosphate (NaPPi) solution for 5 minutes once and with 5% TCA solution for 5 minutes twice at room temperature. After the washes, the filters were washed with 95% ethanol for 5 minutes and then dried under a heat lamp. The washed filters were counted in a standard scintillation cocktail to determine the amount of ³²P in the reaction, as well as the amount of ³²P that was incorporated. The equation used for calculating first-strand synthesis yield is:

$$Amount of cDNA ( pmol ) = \frac{cpm of washed sample}{SA} \times 8 \times 4 ( pmole dNTP / pmole dCTP )$$

The cDNA yield for each reaction at the different time points is shown in the table below (average of two reactions):

| SuperScript ™ | 1 hr | 2 hrs | 4 hrs | 8 hrs |
|---|---|---|---|---|
| II RT (Units) | (pmol) | (pmol) | (pmol) | (pmol) |
| 400 | 941 | 1144 | 1259 | 1272 |
| 800 | 1321 | 1972 | 1992 | 2379 |
| 1000 | 1411 | 1990 | 2402 | 2519 |

### EXAMPLE 7

### cDNA Synthesis with Different Amount of Random Primers

First-Strand cDNA Synthesis Using Different Amounts of Random Primers

We prepared six first-strand cDNA synthesis reactions using total HeLa RNA as starting material. Each reaction was set up using 10 μg of total HeLa RNA primed with 5 μg of oligo(dT)₂₀-VN and 0 ng, 25 ng, 50 ng, 100 ng, 200 ng, or 500 ng of random hexamers. Each mixture was heated to 70° C. for 5 minutes, and then placed on ice for at least 2 minutes.

To each reaction we added a reaction buffer of 50 mM Tris-HCl (pH 8.3), 75 mM KCl, 3 mM M902, 10 mM DTT, 1 μCi ³²P-α-dCTP, 0.5 mM dGTP, 0.5 mM dCTP, 0.35 mM dATP, 0.35 mM dTTP, 0.15 min AA-dUTP, 0.15 mM AH-dATP and 40 Units of RNaseOUT.

SuperScript™ III Reverse Transcriptase (400 Units) was added to each reaction and the reaction volume was brought to 30 μl. Then the reaction was incubated at 46° C. At 2 hours and 4 hours, 5 μl of each reaction were spotted onto a glass fiber (GF/C) filter. The first-strand cDNA yield was calculated by TCA-precipitated ³²P counts, as described below.

TCA Precipitation and First-Strand cDNA Synthesis Calculation

To calculate the specific activity (SA) of ³²P, 2 μl of each reaction were spotted onto

GF/C filter and the cams were counted without TCA wash:

$$SA ( cpm / pmole dCTP ) = \frac{cpm of 2 microliters from unwashed sample}{15000 pmole dCTP} \times 20$$

The GF/C filters containing 5 μl of reaction mixture were washed with ice-cold 10% (w/v) TCA, 1% sodium pyrophosphate (NaPPi) solution for 5 minutes once and with 5% TCA solution for 5 minutes twice at room temperature. After the washes, the filters were washed with 95% ethanol for 5 minutes and then dried under a heat lamp. The washed filters were counted in a standard scintillation cocktail to determine the amount of ³²P in the reaction, as well as the amount of ³²p that was incorporated. The equation used for calculating first-strand synthesis yield is:

$$Amount of cDNA ( pmol ) = \frac{cpm of washed sample}{SA} \times 8 \times 4 ( pmole dNTP / pmole dCTP )$$

The cDNA synthesis yields for the different time points are shown below (average of two reactions):

| Random hexamer (ng) | 2 hrs (pmol) | 4 hrs (pmol) |
|---|---|---|
| 0 | 606 | 591 |
| 25 | 660 | 665 |
| 50 | 706 | 806 |
| 100 | 913 | 988 |
| 200 | 1026 | 1132 |
| 500 | 1115 | 1326 |

### EXAMPLE 8

### Lower Limits of Total RNA Requirement

First-Strand cDNA Synthesis Using Different Amounts of Total RNA

We prepared three first-strand cDNA synthesis reactions using 2 μg, 5 μg, or 10 μg of total HeLa RNA primed with 5 μg of oligo(dT)₂₀-VN. This mixture was heated to 70° C. for 5 minutes, and then placed on ice for at least 2 minutes.

To each reaction we added a reaction buffer of 50 mM Tris-HCl (pH 8.3), 75 mM KCl, 3 mM MgCl₂, 10 mM DTT, 0.5 mM dGTP, 0.5 mM dCTP, 0.35 mM dATP, 0.35 min dTTP, 0.15 mM AA-dUTP, 0.15 mM AH-dATP and 40 Units of RNaseOUT.

SuperScript™ III Reverse Transcriptase (800 Units) was added to each reaction and the reaction volume was brought to 30 μl. Each reaction was then incubated at 46° C. for 2 hours. The reactions were stopped by adding 15 μl of NaOH, then mixed briefly and incubated at 70° C. for 10 min. Fifteen microliters of 1 N HCl were added to neutralize the pH.

Purification of cDNA and Fluorescent Dye Coupling

We added 20 μl of 3 M NaAc, pH 5.2, and 500 μl of Loading Buffer (2.25 M guanidiniun HCl in 70% isopropanol) to each reaction mixture. Each mixture was vortexed briefly, loaded onto a S.N.A.P.™ column, and centrifuged at 14,000×g for 1 minute. We discarded the flowthrough, added 700 μl of wash buffer (100 mM NaCl in 75% ethanol), and centrifuged at 14,000×g for 1 minute. We discarded the flowthrough, and repeated this wash step one more time. Then we centrifuged the column at 14,000×g for 1 minute more to spin down any residual buffer.

We transferred each S.N.A.P.™ column to a new 1.5-ml tube and added 50 μl of dH₂O. We incubated this at room temperature for 1 minute, centrifuged at 14,000×g for 1 minute, and collected the flowthrough. We repeated this elution step one more time and collected the flowthrough. The total volume in each collection tube was about 100 μl.

We added 10 μl of 3M NaAc, pH 5.2, and 2 μl of 20 mg/ml glycogen to each tube, mixed briefly, and then added 300 μl of 100% ethanol. We stored the tubes at −20° C. for at least 30 minutes. We centrifuged each tube at 14,000×g for 10 minutes and carefully discarded the supernatant. We then added 250 μl of 75% ethanol to each tube, mixed gently, and centrifuged at 14,000×g for 2 minutes. We carefully discarded the supernatant and air-dried each pellet for 10 minutes. We then resuspended each pellet in 5 μl of 2× Coupling Buffer (0.1 M Sodium tetraborate, pH 8.5).

We resuspended a pack of Monofunctional Cy3 dye (Amersham, cat #PA23001) in 45 μl of DMSO, and added 5 μl of the Cy3 dye to each cDNA sample in the 2× Coupling Buffer. We mixed briefly and stored the reactions at room temperature in the dark for 1 hour. We then added 5 μl of 4M hydroxylamine and stored the reactions at room temperature in the dark for 15 minutes.

CY3-Labeled cDNA Purification

To purify the dye-labeled cDNA, we added 20 μl of 3M NaAc, pH 5.2, and 500 μl of Loading Buffer to each coupling reaction. We mixed briefly and loaded each labeled cDNA mixture onto a S.N.A.P.™ column. We centrifuged at 14,000×g for 1 minute and discarded the flowthrough. Then we added 700 μl of Wash Buffer, centrifuged at 14,000×g for 1 minute, and discarded the flowthrough. We repeated this wash and spin step one more time, and then performed another centrifugation at 14,000×g for 1 minute to spin down any residual buffer. We transferred each S.N.A.P.™ column to a new 1.5-ml amber tube and added 50 μl of dH₂O. We incubated the columns at room temperature for 1 minute and then centrifuged at 14,000×g for 1 minute, collecting the flowthrough.

### Determining the Amount of Coupled Cy3 and the Ratio of Nucleotide:Dye

The incorporation of Cy3 into the amino-modified cDNA was quantified with UV visible spectroscopy scanning. Cy3 has an absorption maximum at 550 nm. Each tube of column-purified labeled cDNA was scanned at 240-800 nm. The amount of coupled Cy3 and the ratio of nucleotide:dye were calculated as follows:

$$Cy 3 ( pmol ) = \frac{{OD}_{550} - {OD}_{650}}{0.15} \times 50$$

$$cDNA ( pmol ) = \frac{{OD}_{260} - {OD}_{520}}{0.33} \times 40 \times 50$$

$$Ratio = \frac{cDNA ( pmol )}{Cy 3 ( pmol )}$$

The amount of cDNA labeled with Cy3 from 2, 5 and 10 μg of total HeLa RNA is shown below:

| Starting Total RNA | cDNA (pmol) | Cy3 (pmol) | Ratio |
|---|---|---|---|
| 2 μg | 1196 | 31 | 39 |
| 5 μg | 2069 | 60 | 34 |
| 10 μg | 2855 | 72 | 41 |

### EXAMPLE 9

### Optimal Magnesium Concentration for cDNA Synthesis

First-Strand cDNA Synthesis Using Different Magnesium Concentrations

We prepared six first-strand cDNA synthesis reactions: three using 20 μg of total HeLa RNA and three using 50 μg of total HeLa RNA, each primed with 5 μg of oligo(dT)₂₀-VN. Each mixture was heated to 70° C. for 5 minutes, and then placed on ice for at least 2 minutes.

To the reactions containing 20 μg of total HeLa total RNA, we added a reaction buffer of 50 mM Tris-HCl (pH 8.3), 75 mM KCl, 10 mM DTT, 1 μCi ³²P-α-dCTP, 0.5 mM dGTP, 0.5 mM dCTP, 0.5 mM dATP, 0.2 mM dTTP, 0.3 mM AA-dUTP, 40 Units of RNaseOUT™, and either 3 mM, 5 mM, or 8 mM MgCl₂.

To the reactions containing 50 μg of total HeLa RNA, we added a reaction buffer of 50 mM Tris-HCl (pH 8.3), 75 mM KCl, 10 mM DTT, 1 μCi ³²P-α-dCTP, 0.5 mM dGTP, 0.5 mM dCTP, 0.5 mM dATP, 0.2 mM dTTP, 0.3 mM AA-dUTP, 40 Units of RNaseOUT™, and either 3 mM, 5 mM, or 8 mM MgCl₂.

SuperScript™ III Reverse Transcriptase (400 Units) was added to each reaction and the reaction volume was brought to 30 μl. Each reaction was then incubated at 42° C. for 2 hours. The reactions were stopped by adding 10 μl of 0.5 M EDTA. Five microliters of each reaction were spotted onto a glass fiber (GF/C) filter, and the first-strand cDNA yield was calculated by TCA-precipitated ³²P counts, as described below.

TCA Precipitation and First-Strand cDNA Synthesis Calculation

To calculate the specific activity (SA) of ³²P, 2 μl of each sample were spotted onto GF/C filter and the cams were counted without TCA wash:

$$SA ( cpm / pmole dCTP ) = \frac{cpm of 2 microliters from unwashed sample}{15000 pmole dCTP} \times 20$$

The GF/C filters containing 5 μl of reaction mixture were washed with ice-cold 10% (w/v) TCA, 1% sodium pyrophosphate (NaPPi) solution for 5 minutes once and with 5% TCA solution for 5 minutes twice at room temperature. After the washes, the filters were washed with 95% ethanol for 5 minutes and then dried under a heat lamp. The washed filters were counted in a standard scintillation cocktail (Ecolite, ICN, Cat. no. 882475) to determine the amount of ³²p in the reaction, as well as the amount of ³²P that was incorporated. The equation used for calculating first-strand synthesis yield is:

$$Amount of cDNA ( pmol ) = \frac{cpm of washed sample}{SA} \times 8 \times 4 ( pmole dNTP / pmole dCTP )$$

The cDNA yield for each reaction is shown in the table below (average of two reactions):

Synthesized cDNA (pmol)

| MgCl₂ | 20 μg total RNA | 50 μg total RNA |
|---|---|---|
| 3 mM | 2470 | 3069 |
| 5 mM | 2370 | 3391 |
| 8 mM | 1760 | 2639 |

### EXAMPLE 10

### dNTP Concentrations for cDNA Synthesis

First-strand cDNA synthesis using different dNTP concentrations

We prepared three first-strand cDNA synthesis reactions: three using 8.4 μg of total HeLa RNA, each primed with 2 μg of oligo(dT)₂₀-VN. Each mixture was heated to 70° C. for 5 minutes, and then placed on ice for at least 2 minutes.

For the reaction using a 0.5-mM dNTP concentration, we added a reaction buffer of 3 mM MgCl₂, 10 mM DTT, 1 μCi ³²P-α-dCTP, 0.5 mM dGTP, 0.5 mM dCTP, 0.35 mM dATP, 0.35 mM dTTP, 0.35 mM dTTP, 0.15 mM AA-dUTP, 0.15 mM AH-dATP and 40 Units of RNaseOUT.

For the reaction using a 0.75-mM dNTP concentration, we added a reaction buffer of 3 mM MgCl₂, 10 mM DTT, 1 μCi ³²P-α-dCTP, 0.75 mM dGTP, 0.75 mM dCTP, 0.525 mM dATP, 0.525 mM dTTP, 0.225 mM AA-dUTP, 0.225 mM AH-dATP and 40 Units of RNaseOUT.

For the reaction using a 0.75-mM dNTP concentration, we added a reaction buffer of 3 mM MgCl₂, 10 mM DTT, 1 μCi ³²P-α-dCTP, 1 mM dGTP, 1 mM dCTP, 0.7 mM dATP, 0.7 mM dTTP, 0.3 mM AA-dUTP, 0.3 mM AH-dATP and 40 Units of RNaseOUT.

SuperScript™ III Reverse Transcriptase (400 Units) was added to each reaction and the reaction volume was brought to 30 μl. Each reaction was then incubated at 42° C. for 2 hours. The reactions were stopped by adding 10 μl of 0.5 M EDTA. Five microliters of each reaction were spotted onto a glass fiber (GF/C) filter, and the first-strand cDNA yield was calculated by TCA-precipitated ³²P counts, as described below.

TCA Precipitation and First-Strand cDNA Synthesis Calculation

To calculate the specific activity (SA) of ³²P, 2 μl of each sample were spotted onto GF/C filter and the cams were counted without TCA wash:

$$SA ( cpm / pmole dCTP ) = \frac{cpm of 2 microliters from unwashed sample}{15000 pmole dCTP} \times 20$$

The GF/C filters containing 5 μl of reaction mixture were washed with ice-cold 10% (w/v) TCA, 1% sodium pyrophosphate (NaPPi) solution for 5 minutes once and with 5% TCA solution for 5 minutes twice at room temperature. After the washes, the filters were washed with 95% ethanol for 5 minutes and then dried under a heat lamp. The washed filters were counted in a standard scintillation cocktail (Ecolite, ICN, Cat. no. 882475) to determine the amount of ³²P in the reaction, as well as the amount of ³²P that was incorporated. The equation used for calculating first-strand synthesis yield is:

$$Amount of cDNA ( pmol ) = \frac{cpm of washed sample}{SA} \times 8 \times 4 ( pmole dNTP / pmole dCTP )$$

The cDNA yield for each reaction is shown in the table below

| Concentration of dNTP | cDNA yield (pmol) |
|---|---|
| 0.5 | 2477 |
| 0.75 | 3411 |
| 1 | 3746 |

### EXAMPLE 11

### Fluorescent Dyes that were Coupled to cDNA

First-Strand cDNA Synthesis

We prepared three first-strand cDNA synthesis reactions using 0.5 μg of a 0.24-9.5 kb RNA ladder primed with 5 μg of oligo(dT)₂₀-VN. This mixture was heated to 70° C. for 5 minutes, and then placed on ice for at least 2 minutes.

To this reaction, we added a buffer containing 50 mM Tris-HCl (pH 8.3), 75 mM KCl, 3 mM MgCl₂, 10 mM DTT, 0.5 mM dGTP, 0.5 mM dCTP, 0.5 mM dATP, 0.2 mM dTTP, 0.3 min AA-dUTP and 40 Units of RNaseOUT™.

SuperScript™ H Reverse Transcriptase (400 Units) was added to each reaction and the reaction volume was brought to 30 μl. Each reaction was incubated at 42° C. for 2 hours. The reactions were stopped by adding 15 μl of NaOH, then mixed briefly and incubated at 70° C. for 10 min. Fifteen microliters of 1 N HCl were added to neutralize the pH.

Purification of cDNA and Fluorescent Dye Coupling Using Different Dyes

We added 20 μl of 3 M NaAc, pH 5.2, and 500 μl of Loading Buffer (2.25 M guanidiniun HCl in 70% isopropanol) to each reaction mixture. Each mixture was vortexed briefly, loaded onto a S.N.A.P.™ column, and centrifuged at 14,000×g for 1 minute. We discarded the flowthrough, added 700 μl of wash buffer (100 mM NaCl in 75% ethanol), and centrifuged at 14,000×g for 1 minute. We discarded the flowthrough, and repeated this wash step one more time. Then we centrifuged the column at 14,000×g for 1 minute more to spin down any residual buffer.

We transferred each S.N.A.P.™ column to a new 1.5-ml tube and added 50 μl of dH₂O. We incubated this at room temperature for 1 minute, centrifuged at 14,000×g for 1 minute, and collected the flowthrough. We repeated this elution step one more time and collected the flowthrough. The total volume in each collection tube was about 100 μl.

We added 10 μl of 3M NaAc, pH 5.2, and 2 μl of 20 mg/ml glycogen to each tube, mixed briefly, and then added 300 μl of 100% ethanol. We stored the tubes at −20° C. for at least 30 minutes. We centrifuged each tube at 14,000×g for 10 minutes and carefully discarded the supernatant. We then added 250 μl of 75% ethanol to each tube, mixed gently, and centrifuged at 14,000×g for 2 minutes. We carefully discarded the supernatant and air dried each pellet for 10 minutes. We then resuspended each pellet in 5 μl of 2× Coupling Buffer (0.1 M Sodium tetraborate, pH 8.5).

For Cy3, we resuspended a pack of Monofunctional Cy3 dye (Amersham, cat #PA23001) in 45 μl of DMSO, and added 5 μl of the Cy3 dye to each cDNA sample. We mixed briefly and stored the reaction at room temperature in the dark for 1 hour.

For Alexa 546, we resuspended 1 mg of Alexa™ 546 (Molecular probe, Cat #A-20002) in 50 μl of DMSO, and added 5 μl of the dye to each cDNA sample. We mixed briefly and stored the reaction at room temperature in the dark for 1 hour.

For Oyster™ 556, we resuspended a pack of Monofunctional Oyster™ 556 dye (Denovo Biolabels, Cat #OY-556-1XO.2) in 45 μl of DMSO. We mixed briefly and stored the reaction at room temperature in the dark for 1 hour.

We then added 5 μl of 4M hydroxylamine to each reaction and stored the reactions at room temperature in the dark for 15 minutes.

Labeled cDNA Purification

To purify the dye-labeled cDNA, we added 20 μl of 3M NaAc, pH 5.2, and 500 μl of Loading Buffer to each coupling reaction. We mixed briefly and loaded each labeled cDNA mixture onto a S.N.A.P.™ column. We centrifuged at 14,000×g for 1 minute and discarded the flowthrough. Then we added 700 μl of Wash Buffer, centrifuged at 14,000×g for 1 minute, and discarded the flowthrough. We repeated this wash and spin step one more time, and then performed another centrifugation at 14,000×g for 1 minute to spin down any residual buffer. We transferred each S.N.A.P.™ column to a new 1.5-ml amber tube and added 50 μl of dH₂O. We incubated the columns at room temperature for 1 minute and then centrifuged at 14,000×g for 1 minute, collecting the flowthrough.

### Determining the Amount of Coupled Dye

The incorporation of dye into the amino-modified cDNA was quantified with UV visible spectroscopy scanning. Each tube of column-purified labeled cDNA was scanned at 240800 nm. The amount of coupled dye was calculated as follows:

$$Cy 3 ( pmol ) = \frac{{OD}_{550} - {OD}_{650}}{0.15} \times 50$$

$$\begin{matrix}Alexa 546 ( pmol ) = \frac{{OD}_{554} - {OD}_{650}}{0.104} \times 50 \\ Oyster 556 ( pmol ) = \frac{{OD}_{556} - {OD}_{650}}{0.155} \times 50\end{matrix}$$

The results for the average of two reactions per dye are shown below:

| Dye type | Dy (pmol) |
|---|---|
| Cy3 | 29 |
| Alexa 546 | 33 |
| Oyster 556 | 19 |

### EXAMPLE 12

Cy3 Coupled to cDNA with a Different Coupling Buffer

First-Strand cDNA Synthesis

We prepared four first-strand cDNA synthesis reactions using 0.5 μg of a 0.24-9.5 kb RNA ladder primed with 5 μg of oligo(dT)₂₀-VN. This mixture was heated to 70° C. for 5 minutes, and then placed on ice for at least 2 minutes.

To each reaction, we added a buffer containing 50 mM Tris-HCl (pH 8.3), 75 mM KCl, 3 mM MgCl₂, 10 mM DTT, 0.5 mM dGTP, 0.5 mM dCTP, 0.5 mM dATP, 0.2 mM dTTP, 0.3 mM AA-dUTP and 40 Units of RNaseOUT™.

SuperScript™ II Reverse Transcriptase (400 Units) was added to each reaction and the reaction volume was brought to 30 μl. Each reaction was incubated at 42° C. for 2 hours. The reactions were stopped by adding 15 μl of NaOH, then mixed briefly and incubated at 70° C. for 10 min. Fifteen microliters of 1 N HCl were added to neutralize the pH.

Purification of cDNA and Fluorescent Dye Coupling Using Different Dyes

We added 20 μl of 3 M NaAc, pH 5.2, and 500 μl of Loading Buffer (2.25 M guanidiniun HCl in 70% isopropanol) to each reaction mixture. Each mixture was vortexed briefly, loaded onto a S.N.A.P.™ column, and centrifuged at 14,000×g for 1 minute. We discarded the flowthrough, added 700 μl of wash buffer (100 mM NaCl in 75% ethanol), and centrifuged at 14,000×g for 1 minute. We discarded the flowthrough, and repeated this wash step one more time. Then we centrifuged the column at 14,000×g for 1 minute more to spin down any residual buffer.

We transferred each S.N.A.P.™ column to a new 1.5-ml tube and added 50 μl of dH₂O. We incubated this at room temperature for 1 minute, centrifuged at 14,000×g for 1 minute, and collected the flowthrough. We repeated this elution step one more time and collected the flowthrough. The total volume in each collection tube was about 100 μl.

We added 10 μl of 3M NaAc, pH 5.2, and 2 μl of 20 mg/ml glycogen to each tube, mixed briefly, and then added 300 μl of 100% ethanol. We stored the tubes at −20° C. for at least 30 minutes. We centrifuged each tube at 14,000×g for 10 minutes and carefully discarded the supernatant. We then added 250 μl of 75% ethanol to each tube, mixed gently, and centrifuged at 14,000×g for 2 minutes. We carefully discarded the supernatant and air dried each pellet for 10 minutes.

Two pellets were then resuspended in 5 μl of 2× Coupling Buffer (0.1 M Sodium tetraborate, pH 8.5), while another two were resuspended in 0.1 M sodium bicarbonate, pH 9.0.

We resuspended a pack of Monofunctional Cy3 dye (Amersham, cat #PA23001) in 45 μl of DMSO, and added 5 μl of the Cy3 dye to each cDNA sample. We mixed briefly and stored the reactions at room temperature in the dark for 1 hour. We then added 5 μl of 4M hydroxylamine and stored the reactions at room temperature in the dark for 15 minutes.

Cy3-Labeled cDNA Purification

To purify the dye-labeled cDNA, we added 20 μl of 3M NaAc, pH 5.2, and 500 μl of Loading Buffer to each coupling reaction. We mixed briefly and loaded each labeled cDNA mixture onto a S.N.A.P.™ column. We centrifuged at 14,000×g for 1 minute and discarded the flowthrough. Then we added 700 μl of Wash Buffer, centrifuged at 14,000×g for 1 minute, and discarded the flowthrough. We repeated this wash and spin step one more time, and then performed another centrifugation at 14,000×g for 1 minute to spin down any residual buffer. We transferred each S.N.A.P.™ column to a new 1.5-ml amber tube and added 50 μl of dH₂O. We incubated the columns at room temperature for 1 minute and then centrifuged at 14,000×g for 1 minute, collecting the flowthrough.

### Determining the Amount of Coupled Cy3 and the Ratio of Nucleotide:Dye

The incorporation of Cy3 into the amino-modified cDNA was quantified with UV visible spectroscopy scanning. Cy3 has an absorption maximum at 550 nm. Each tube of column-purified labeled cDNA was scanned at 240-800 nm. The amount of coupled Cy3 and the ratio of nucleotide:dye were calculated as follows:

$$Cy 3 ( pmol ) = \frac{{OD}_{550} - {OD}_{650}}{0.15} \times 50$$

$$cDNA ( pmol ) = \frac{{OD}_{260} - {OD}_{520}}{0.33} \times 40 \times 50$$

$$Ratio = \frac{cDNA ( pmol )}{Cy 3 ( pmol )}$$

The results for an average of two reactions are shown below:

| Buffer | cDNA (pmol) | Cy3 (pmol) | Ratio |
|---|---|---|---|
| 0.1M sodium tetraborate, pH 8.5 | 440 | 29 | 46 |
| 0.1M sodium bicarbonate, pH 9.0 | 370 | 13 | 84 |

### EXAMPLE 13

### Purification of Fluorescence-Labeled cDNA

First-Strand cDNA Synthesis

We prepared three first-strand cDNA synthesis reactions using 0.5 μg of total HeLa RNA primed with 5 μg of oligo(dT)₂₀-VN. This mixture was heated to 70° C. for 5 minutes, and then placed on ice for at least 2 minutes.

To each reaction, we added a buffer of 50 mM Tris-HCl (pH 8.3), 75 mM KCl, 3 mM MgCl₂, 10 mM DTT, 1 μCi ³²P-α-dCTP, 0.5 mM dGTP, 0.5 mM dCTP, 0.35 mM dATP, 0.35 mM dTTP, 0.15 mM AA-dUTP, 0.15 mM. AH-dATP, and 40 Units of RNaseOUT™.

SuperScript™ III Reverse Transcriptase (400 Units) was added to each reaction and the reaction volume was brought to 30 μl. Each reaction was incubated at 46° C. for 2 hours. The reactions were stopped by adding 15 μl of NaOH, then mixed briefly and incubated at 70° C. for 10 min. Fifteen microliters of I N HO were added to neutralize the pH.

Purification of cDNA and Fluorescent Dye Coupling

We added 20 μl of 3 M NaAc, pH 5.2, and 500 μl of Loading Buffer (2.25 M guanidiniun HCl in 70% isopropanol) to each reaction mixture. Each mixture was vortexed briefly, loaded onto a S.N.A.P.™ column, and centrifuged at 14,000×g for 1 minute. We discarded the flowthrough, added 700 μl of wash buffer (100 mM NaCl in 75% ethanol), and centrifuged at 14,000×g for 1 minute. We discarded the flowthrough, and repeated this wash step one more time. Then we centrifuged the column at 14,000×g for 1 minute more to spin down any residual buffer.

We transferred each S.N.A.P.™ column to a new 1.5-ml tube and added 50 μl of dH₂O. We incubated this at room temperature for 1 minute, centrifuged at 14,000×g for 1 minute, and collected the flowthrough. We repeated this elution step one more time and collected the flowthrough. The total volume in each collection tube was about 100 μl.

We added 10 μl of 3M NaAc, pH 5.2, and 2 μl of 20 mg/ml glycogen to each tube, mixed briefly, and then added 300 μl of 100% ethanol. We stored the tubes at −20° C. for at least 30 minutes. We centrifuged each tube at 14,000×g for 10 minutes and carefully discarded the supernatant. We then added 250 μl of 75% ethanol to each tube, mixed gently, and centrifuged at 14,000×g for 2 minutes. We carefully discarded the supernatant and air dried each pellet for 10 minutes. We then resuspended each pellet in 5 μl of 2× Coupling Buffer (0.1 M Sodium tetraborate, pH 8.5).

We resuspended a pack of Monofunctional Cy5™ dye (Amersham, cat #PA25001) in 45 μl of DMSO, and added 5 μl of the Cy5™ dye to each cDNA sample in the 2× Coupling Buffer. We mixed briefly and stored the reactions at room temperature in the dark for 1 hour. We then added 5 μl of 4M hydroxylamine and stored the reactions at room temperature in the dark for 15 minutes.

Cy5-Labeled cDNA Purification Using Different Columns

We pooled all the reactions and aliquoted 15 μl of the mixture into three 1.5-ml tubes. We added 20 μl of 3M NaAc, pH 5.2, and 500 μl of Loading Buffer to each tube and mixed briefly. Then we loaded each mixture onto either a S.N.A.P.™ column, MBP column (new), or a MinElute column (Qiagen, Cat #28204). We centrifuged at 14,000×g for 1 minute and discarded the flowthrough. Then we added 700 μl of Wash Buffer, centrifuged at 14,000×g for 1 minute, and discarded the flowthrough. We repeated this wash and spin step one more time, and then performed another centrifugation at 14,000×g for 1 minute to spin down any residual buffer. We transferred each column to a new 1.5-ml amber tube and added 50 μl of dH₂O, We incubated the columns at room temperature for 1 minute and then centrifuged at 14,000×g for 1 minute, collecting the flowthrough.

### Determining the Amount of Coupled Cy5

The incorporation of Cy5 into the amino-modified cDNA was quantified with UV visible spectroscopy scanning. Cy5 has an absorption maximum at 650 nm. Each tube of column-purified labeled cDNA was scanned at 240-800 nm. The amount of coupled Cy5 was calculated as follows:

$$Cy 5 ( pmol ) = \frac{{OD}_{650} - {OD}_{750}}{0.25} \times 50$$

The amount of cDNA labeled with Cy5 using the different columns is shown below:

| Column | Cy5 (pmol) |
|---|---|
| SNAP | 45 |
| MBP | 19 |
| MinElute | 52 |

### EXAMPLE 14

### Dye Coupling Without Hydroxylamine Quench Step

First-Strand cDNA Synthesis

We prepared two first-strand cDNA synthesis reactions using 10 μg of total HeLa RNA primed with 5 μg of oligo(dT)₂₀-VN. This mixture was heated to 70° C. for 5 minutes, and then placed on ice for at least 2 minutes.

To each reaction, we added a buffer of 50 mM Tris-HCL (pH 8.3), 75 mM KCl, 3 mM MgCl₂, 10 mM DTT, 1 μCi ³²P-α-dCTP, 0.5 mM dGTP, 0.5 mM dCTP, 0.35 mM dATP, 0.35 mM dTTP, 0.15 mM AA-dUTP, 0. 15 mM AH-dATP, and 40 Units of RNaseOUT™.

SuperScript™ III Reverse Transcriptase (800 Units) was added to each reaction and the reaction volume was brought to 30 μl. Each reaction was incubated at 46° C. for 2 hours. The reactions were stopped by adding 15 μl of NaOH, then mixed briefly and incubated at 70° C. for 10 min. Fifteen microliters of 1 N HCl were added to neutralize the pH.

Purification of cDNA and Fluorescent Dye Coupling

We added 20 μl of 3 M NaAc, pH 5.2, and 500 μl of Loading Buffer (2.25 M guanidiniun HCl in 70% isopropanol) to each reaction mixture. Each mixture was vortexed briefly, loaded onto a S.N.A.P.™ column, and centrifuged at 14,000×g for 1 minute. We discarded the flowthrough, added 700 μl of wash buffer (100 mM NaCl in 75% ethanol), and centrifuged at 14,000×g for 1 minute. We discarded the flowthrough, and repeated this wash step one more time. Then we centrifuged the column at 14,000×g for 1 minute more to spin down any residual buffer.

We transferred each S.N.A.P.™ column to a new 1.5-ml tube and added 50 μl of dH₂O. We incubated this at room temperature for I minute, centrifuged at 14,000×g for 1 minute, and collected the flowthrough. We repeated this elution step one more time and collected the flowthrough. The total volume in each collection tube was about 100 μl.

We added 10 μl of 3M NaAc, pH 5.2, and 2 μl of 20 mg/ml glycogen to each tube, mixed briefly, and then added 300 μl of 100% ethanol. We stored the tubes at −20° C. for at least 30 minutes. We centrifuged each tube at 14,000×g for 10 minutes and carefully discarded the supernatant. We then added 250 μl of 75% ethanol to each tube, mixed gently, and centrifuged at 14,000×g for 2 minutes. We carefully discarded the supernatant and air dried each pellet for 10 minutes. We then resuspended each pellet in 5 μl of 2× Coupling Buffer (0.1 M Sodium tetraborate, pH 8.5).

We resuspended a pack of Monofunctional Cy5Tm dye (Amersham, cat #PA25001) in 45 μl of DMSO, and added 5 μl of the Cy5™ dye to each cDNA sample in the 2× Coupling Buffer. We mixed briefly and stored the reactions at room temperature in the dark for 1 hour.

For the reaction without the hydroxylamine step, we went directly to the purification step. For the reaction with the hydroxylamine quench step, we added 5 μl of 4M hydroxylamine and stored the reactions at room temperature in the dark for 15 minutes.

Cy5-Labeled cDNA Purification Using Different Columns

To purify the dye-labeled cDNA, we added 20 μl of 3M NaAc, pH 5.2, and 500 μl of Loading Buffer to each coupling reaction. We mixed briefly and loaded each labeled cDNA mixture onto a S.N.A.P.™ column. We centrifuged at 14,000×g for 1 minute and discarded the flowthrough. Then we added 700 μl of Wash Buffer, centrifuged at 14,000×g for 1 minute, and discarded the flowthrough. We repeated this wash and spin step one more time, and then performed another centrifugation at 14,000×g for 1 minute to spin down any residual buffer. We transferred each S.N.A.P.™ column to a new 1.5-ml amber tube and added 50 μl of dH₂O. We incubated the columns at room temperature for 1 minute and then centrifuged at 14,000×g for 1 minute, collecting the flowthrough,

### Determining the Amount of Coupled Cy5

The incorporation of Cy5 into the amino-modified cDNA was quantified with UV visible spectroscopy scanning. Cy5 has an absorption maximum at 650 nm. Each tube of column-purified labeled cDNA was scanned at 240-800 nm. The amount of coupled Cy5 was calculated as follows:

$$Cy 5 ( pmol ) = \frac{{OD}_{650} - {OD}_{750}}{0.25} \times 50$$

The amount of cDNA labeled with Cy5 with and without the quench step is shown below:

| | Cy5 (pmol) |
|---|---|
| With hydroxylamine | 57 |
| Without hydroxylamine | 88 |

### EXAMPLE 15

### Labeling with Alexa Dyes

cDNA Synthesis

We prepared four first-strand cDNA synthesis reactions using total

RNA as starting material. Each reaction was set up using 10 μg of total Hela RNA primed with 5 μg of oligo(dT)₂₀-VN. This mixture was heated to 70° C. for 5 minutes, and then placed on ice for at least 2 min.

To each reaction we added a reaction buffer of 50 mM Tris-HCL (pH 8.3), 75 mM KCl, 3 mM MgCl₂, 10 mM DTT, 0.5 mM dGTP, 0.5 mM dCTP, 0.35 mM dATP, 0.35 mM dTTP, 0. 15 mM AA-dUTP, 0.15 mM AH-dATP and 40 Units of RNaseOUT.

SuperScript™ III Reverse Transcriptase (800 Units) was added to each reaction and the reaction volume was brought to 30 Each reaction was then incubated at 46° C. for 2 hours. The reactions were stopped by adding 15 μl of NaOH, then mixed briefly and incubated at 70° C. for 10 min. Fifteen microliters of 1 N HCl were added to neutralize the pH.

Purification of cDNA and Fluorescent Dye Coupling

We added 20 μl of 3 M NaAc, pH 5.2, and 500 μl of Loading Buffer (2.25 M guanidiniun HCl in 70% isopropanol) to each reaction mixture. Each mixture was vortexed briefly, loaded onto a S.N.A.P.™ column, and centrifuged at 14,000×g for 1 minute. We discarded the flowthrough, added 700 μl of wash buffer (100 mM NaCl in 75% ethanol), and centrifuged at 14,000×g for 1 minute. We discarded the flowthrough, and repeated this wash step one more time. Then we centrifuged the column at 14,000×g for 1 minute more to spin down any residual buffer.

We transferred each S.N.A.P.™ column to a new 1.5-ml tube and added 50 μl of dH₂O. We incubated this at room temperature for 1 minute, centrifuged at 14,000×g for 1 minute, and collected the flowthrough. We repeated this elution step one more time and collected the flowthrough. The total volume in each collection tube was about 100 μl.

We added 10 μl of 3M NaAc, pH 5.2, and 2 μl of 20 mg/ml glycogen to each tube, mixed briefly, and then added 300 μl of 100% ethanol. We stored the tubes at −20° C. for at least 30 minutes. We centrifuged each tube at 14,000×g for 10 minutes and carefully discarded the supernatant. We then added 250 μl of 75% ethanol to each tube, mixed gently, and centrifuged at 14,000×g for 2 minutes. We carefully discarded the supernatant and air-dried each pellet for 10 minutes. We then resuspended each pellet in 5 μl of 2× Coupling Buffer (0.1 M Sodium tetraborate, pH 8.5).

Resuspended 1 mg of Monofunctional Alexa Fluor 546 (Molecular probe A-20002, lot #50B5-1) in 112.5 μl of DMSO. Added 5 μl of Alexa 546 to two cDNA samples in 2× coupling buffer. Mixed briefly and kept at room temperature in the dark for 1 hour. Resuspended 1 mg of Monofunctional Alexa Fluor 647 (Molecular probe A-20006, lot #50B7-1) in 112.5 μl of DMSO. Added 5 μl of Alexa 647 to two cDNA samples in 2× coupling buffer. Mixed briefly and kept at room temperature in the dark for 1 hour.

Alexa-Labeled cDNA Purification

Added 20 μl 3M NaAc, pH 5.2 and 500 μl of Loading Buffer to the coupling reaction. Mixed briefly and loaded CyDye-cDNA mixture onto the S.N.A.P.™ column. Centrifuged at 14,000 rpm for 1 min. Discarded the flowthrough, and added 700 μl of Wash Buffer. Centrifuged at 14,000 rpm for 1 minute. Discarded the flowthrough. Added another 700 μl of Wash Buffer. Centrifuged at 14,000 rpm for 1 min. Discard the flowthrough. Centrifuged at 14,000 rpm for 1 minute to spin down any residual buffer. Transferred the SNAP column to a new 1.5 ml amber tube and added 50 μl dH₂O. Incubated at room temperature for 1 min. Centrifuged at 14,000 rpm for 1 minute.

Determination of the Amount of Coupled Allexa into cDNA and Ratio of Nucleotide:Dye

The incorporation of Alexa into aminoallyl-modified cDNA was quantified with UV visible spectroscopy scanning. Alexa546 have absorption maximum at 556 nm. Alexa647 have absorption maximum at 650 nm. The SNAP column purified cDNAs were scanned at 240 nm-800 nm. The calculations is as the following:

$$Alexa 546 ( pmol ) = \frac{{OD}_{556} - {OD}_{650}}{0.104} \times 50$$

$$Alexa 647 ( pmol ) = \frac{{OD}_{650} - {OD}_{750}}{0.239} \times 50$$

$$cDNA ( pmol ) = \frac{{OD}_{260} - {OD}_{520}}{0.33} \times 40 \times 50$$

$$Ratio = \frac{cDNA ( pmol )}{Alexa Dye ( pmol )}$$

cDNA synthesized from 10 μg Hela total RNA labeled with Alexa dye, the result is the average of two reactions:

| | cDNA (pmol) | Dye (pmol) | Ratio |
|---|---|---|---|
| Alexa 546 | 2395 ± 114 | 206 ± 16 | 12 |
| Alexa 647 | 1600 ± 283 | 136 ± 8 | 12 |

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

1.-43. (canceled)

44. A method of synthesizing one or more nucleic acid molecules comprising incubating one or more nucleic acid templates with two or more modified nucleotides under conditions sufficient to make one or more first nucleic acid molecules complementary to all or a portion of said one or more templates, wherein at least one said nucleic acid molecule contains said two or more modified nucleotides incorporated therein.

45. The method of claim 44, wherein at least one of said modified nucleotides is selected from the group consisting of aminoallyl-dUTP and aminohexyl-dATP.

46. (canceled)

47. The method of claim 44, wherein said nucleic acid template is mRNA or a population of mRNA molecules.

48. The method of claim 44, further comprising incubating said one or more nucleic acid molecules under conditions sufficient to make one or more second nucleic acid molecules complementary to all or a portion of said one or more first nucleic acid molecules.

49. The method of claim 44, further comprising incubating said one or more nucleic acid molecules in the presence of one or more fluorescent labels under conditions sufficient to couple one or more of said labels to at least one of said modified nucleotides incorporated therein.

50. (canceled)

51. The method of claim 49, wherein at least one of said one or more fluorescent labels is a cyanine dye.

52. (canceled)

53. (canceled)

54. The method of claim 49, wherein at least one of said one or more fluorescent labels is an Alexa dye.

55. A kit comprising one or more enzymes having reverse transcriptase activity, and one or more non-naturally occurring fluorescent labels, and two or more different, monomeric modified nucleotides for incorporation into a first nucleic acid molecule, wherein said modified nucleotides have the ability to bind to the one or more fluorescent labels; and at least one of the two or more modified nucleotides is aminoallyl-dUTP.

56. (canceled)

57. The kit of claim 55, wherein at least two of said modified nucleotides contain primary reactive amines.

58.-60. (canceled)

61. The kit of claim 55, wherein at least two of said modified nucleotides is selected from the group consisting of aminoallyl-dUTP and aminohexyl-dATP.

62. The kit of claim 55, further comprising at least one nucleic acid template.

63. The kit of claim 62, wherein said nucleic acid template is DNA.

64. The kit of claim 62, wherein said nucleic acid template is RNA.

65. The kit of claim 64, wherein said RNA template is a mRNA or a

66. (canceled)

67. (canceled)

68. The kit of claim 55, wherein at least one of the fluorescent labels is a cyanine dye.

69. The kit of claim 68, wherein the cyanine dye is selected from the group consisting of Cy3 and/or Cy5.

70. The kit of claim 55, wherein at least one of the fluorescent labels is an Alexa dye.

71. (canceled)

72. The kit of claim 55, wherein said two or more modified nucleotides are in a mixture in a first container.

73. The kit of claim 72, wherein said one or more enzymes having reverse transcriptase activity are in a mixture in a second container.

74. The kit of claim 55, wherein said one or more enzymes having reverse transcriptase activity comprises a Moloney Murine Leukemia Virus reverse transcriptase (M-MLV RT).
